SEQUENCE LISTING (1 sequence)

SEQ ID NO: 1 - protein, 114 aa, mammalian
SPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

(19) United States
(12) Patent Application Publication
(10) Pub. No.: US 2008/0206139 A1
(43) Pub. Date: Aug. 28, 2008

(54) DELIVERY SYSTEM FOR DIAGNOSTIC AND THERAPEUTIC AGENTS

(75) Inventors: James R. Connor, Hershey, PA (US); A. B. Madhankumar, Hershey, PA (US)

Correspondence Address: AKERMAN SENTERFITT, P.O. BOX 3188, WEST PALM BEACH, FL, 33402-3188, US

(73) Assignee: The Penn State Research Foundation, University Park, PA (US)

(21) Appl. No.: 11/556,574

(22) Filed: Nov. 3, 2006

(51) Int. Cl. A61K 49/00; A61K 9/127; A61P 35/00

(52) U.S. Cl. 424/9.1; 424/450

(57) ABSTRACT

Nanovesicles are specifically targeted to abnormal cells. The targeting moiety is conjugated to the nanovesicle which comprises a therapeutic composition. These nanovesicles are useful in treatment of a wide spectrum of disorders.

### CROSS-REFERENCE TO RELATED APPLICATION

The present application claims the priority of U.S. Ser. No. 11/584,122 entitled “DELIVERY SYSTEM FOR DIAGNOSTIC AND THERAPEUTIC AGENTS,” filed Oct. 20, 2006 and U.S. provisional patent application No. 60/728,654, entitled “DELIVERY SYSTEM FOR DIAGNOSTIC AND THERAPEUTIC AGENTS,” filed Oct. 20, 2005, which are incorporated herein by reference in their entirety.

### FIELD OF THE INVENTION

This invention relates to the treatment of tumors and other diseases using specifically targeted nanovesicles comprising therapeutic compositions.

### BACKGROUND

Specific targeting of diagnostic and therapeutic agents to cells and tissues is highly desirable in both medical and research settings. Although many delivery systems for diagnostic and therapeutic agents have been generated, an effective and specific delivery system with minimal side effects and low toxicity has remained elusive. Thus, there is a continuing need for diagnostic and therapeutic agent delivery technology which achieves these goals.

The brain is an exceptionally challenging target for medical treatment and diagnosis. In particular, the brain is unique among organs in comprising many cell types having various functions. Further, the blood brain barrier inhibits the effectiveness of systemic administration of diagnostic and therapeutic agents for delivery to the brain. The blood-brain barrier (BEB) represents a formidable obstacle for delivering therapeutic and diagnostic agents to central nervous system targets. Several lipophilic, therapeutic drugs such as doxorubicin have proven to be actively effluxed by P-glycoprotein (Pgp) expressed at the luminal membrane of the brain capillary endothelial cells, resulting in the very low apparent blood-brain barrier (BBB) permeation of these Pgp substrates from the blood circulating to the brain. Compositions and methods for effective delivery of a therapeutic agent and/or a diagnostic agent across the blood brain barrier to a central nervous system (CNS) target are needed.

### SUMMARY

A delivery system is provided according to the present invention which includes a delivery vehicle for a cargo moiety such as a diagnostic and/or therapeutic agent. The delivery vehicle is capable of crossing the blood brain barrier and delivering a cargo moiety to the CNS.

A delivery vehicle included in an embodiment of a system according to the invention includes particles capable of association with a cargo moiety for delivery of the cargo to a target. A particle is capable of association with a cargo moiety where association does not inactivate a desired function of the cargo moiety and where the cargo moiety may be transported along with the particle to a desired location. Such particles include microspheres, nanoparticles, micelles, niosomes and liposomes for instance.

An important advantage and benefit of the instant invention is that the chemotherapeutic agent is target to the diseased area and delivered to the desired abnormal cells and cell mass. This specific targeting avoids the need for whole body chemotherapy and/or radiation therapy, thereby avoiding the associated disadvantages of whole body treatments.

In a preferred embodiment, a pharmaceutical delivery system comprises a particulate delivery vehicle having a wall, the wall defining an external surface and an internal volume; and, a cargo moiety associated with the delivery vehicle.

In a preferred embodiment, the delivery vehicle is a liposome. In one aspect, the cargo moiety is at least partially localized in the internal volume of the delivery vehicle.

In another preferred embodiment, a targeting moiety is conjugated to the external surface of the wall of the delivery vehicle. Preferably, the targeting moiety is a ligand of a receptor present on a target cell and the receptor is preferentially expressed by a target cell compared to a non-target cell. In one aspect, the receptor is a human IL-13Rα2 receptor and the targeting moiety is human IL-13.

In another preferred embodiment, the targeting moiety is a mutant of IL-13 which binds a human IL-13α2 receptor. Preferably, the target cell is a tumor cell. In one aspect, the mutant of IL-13 binds a human IL-13Rα2 receptor binds to the IL-13Rα2 receptor with greater affinity than it binds to a wild-type human IL-13 receptor.

In another preferred embodiment, the mutant of IL-13 is selected from the group consisting of: IL-13.K105R, IL-13.E13K and a combination thereof.

In another preferred embodiment, the delivery vehicle has a diameter in the range of about 1-1000 nanometers. Preferably, the delivery vehicle has a diameter in the range of about 50-150 nanometers.

In a preferred embodiment, the cargo moiety comprises anti-tumor agents or other pharmaceutical compositions for delivery to abnormal cells, i.e. any cells which do not function according to the physiological norm similarly situated like cells, such as cells infected with a biological organism, tumor cells, and the like. The cargo moiety comprises: iron; and/or an anti-cancer composition; and/or an siRNA composition, such as for example, an anti-ferritin siRNA composition.

In another preferred embodiment, a pharmaceutical composition comprises a plurality of particulate delivery vehicles, each particulate delivery vehicle having a wall defining an external surface and an internal volume, and each particulate delivery vehicle having a cargo moiety associated therewith and a targeting moiety conjugated thereto; and, a pharmaceutically acceptable carrier. Preferably, the plurality of particle delivery vehicles has a mean particle size in the range of about 1-1000 nanometers.

In a preferred embodiment, the plurality of particle delivery vehicles has a mean particle size in the range of about 50-150 nanometers.

In another preferred embodiment, a targeting moiety is selected from the group consisting of: human IL-13, an IL-13.K105R mutant of human IL-13, an IL-13.E13K mutant of human IL-13, and a combination thereof.

In another preferred embodiment, a liposome comprises a human wild-type IL-13 or a mutant of human wild-type IL-13 having higher affinity for the human IL-13Rα2 receptor than wild-type IL-13 conjugated thereto, the liposome encapsulating an anti-cancer drug.

In another preferred embodiment, a method of treating and/or diagnosing an actual or suspected CNS disorder in an individual, comprises administering a therapeutically effective amount of a pharmaceutical composition comprising a plurality of particulate delivery vehicles, each associated with a cargo moiety which is a therapeutic and/or diagnostic agent, wherein the association of the therapeutic and/or diagnostic agent with the plurality of particulate delivery vehicles facilitates passage of the therapeutic and/or diagnostic agent through the blood brain barrier into the CNS such that the actual or suspected CNS disorder is treated and/or diagnosed. Preferably, the particulate delivery vehicles further comprise a targeting moiety, wherein the targeting moiety comprises IL-13 and/or a mutant thereof. In one aspect, the targeting moiety comprises an IL-13.K105R mutant of human IL-13, and/or an IL-13.E13K mutant of human IL-13. The cargo moiety comprises: iron; and/or an anti-cancer composition; and/or an siRNA composition, such as for example, an anti-ferritin siRNA composition.

In another preferred a pharmaceutical composition comprises a particulate delivery vehicle having a wall, the wall defining an external surface and an internal volume; and, a cargo moiety associated with the delivery vehicle.

In another preferred embodiment, a pharmaceutical composition comprises a liposome; targeting moiety; and, a chemotherapeutic agent. Preferably the liposome comprises DPPC:CHOL:DSPE-PEG:PDP-SA in a ratio of 10:5:1.5:1.5 and is about 20 nm to about 220 nm in size.

In another embodiment the targeting moiety is attached to the liposome via a thiolated group.

In another preferred embodiment, a method of treating a cancer patient comprises administering to the patient a composition comprising pharmaceutical composition comprising: a liposome; targeting moiety; and, a chemotherapeutic agent; and, treating a cancer patient.

In another preferred embodiment, the pharmaceutical composition can be administered in conjunction with chemotherapy and radio therapy.

Other aspects of the invention are described infra.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIG. 1 is a graph showing the neutralization potential of the wild type IL-13 and its mutants at variable concentrations of the protein and at a fixed concentration of cytotoxin IL13-PE38QQR (10 ng/ml).

FIG. 2A is a schematic illustration showing PEG liposomes conjugated to the IL-13 and Tf molecule. FIGS. 2B and 2C is a scan of a TEM picture of IL-13 conjugated liposomes after staining with uranyl acetate (particle size range=50-200 nm).

FIG. 3 is a scan of photographs showing the binding and internalization of IL-13 conjugated rhodamine labeled liposomes on various glioma and normal cells.

FIGS. 4A-4C are scans of photographs showing intrinsic fluorescence of doxorubicin (DXR) delivered to U251 glioma cells. FIG. 4A Free doxorubicin; FIG. 4B DXR encapsulated in unconjugated liposomes, and FIG. 4C DXR encapsulated in IL-13 conjugated liposomes.

FIG. 5 are scans of photographs showing the binding of rhodamine PE-labeled IL-13 conjugated liposomes on various brain tumor sections and in normal brain indicated that higher specific binding is observed in Glioblastoma Multiforme (GBM) which overexpress IL-13Rα2 receptor. The binding pattern also shows that liposomes binds specifically to certain low grade astrocytomas.

FIG. 6 is a scan of photographs showing the intrinsic fluorescence of doxorubicin encapsulated IL-13 conjugated (targeted) and unconjugated (non-targeted) liposomes on tumor sections.

FIG. 7 is a graph showing results from a cytotoxicity assay of IL-13 and Tf conjugated liposomes carrying doxorubicin towards U251 cells. The cytotoxic potential of ligand targeted liposomes are higher than the unconjugated liposomes carrying the same amount of doxorubicin. This observation demonstrates that receptor mediated endocytosis of IL-13 conjugated liposomes results in enhanced delivery of the encapsulated doxorubicin resulting in higher cytotoxicity.

FIG. 8 is a graph showing cytotoxicity experiments performed with media from a blood brain barrier transport chamber experiment.

FIG. 9 is a graph showing the therapeutic efficacy of the IL-13 receptor targeted liposomes carrying doxorubicin was tested in a subcutaneous glioma tumor model in nude mice. Mice were given intraperitoneal injections once a week. The insert shows that mice receiving targeted liposomes with doxorubicin had a greater reduction in tumor size in the first two weeks compared to the animals receiving the same concentration of unconjugated liposomes and doxorubicin. The tumors of the other groups increased during the initial three weeks of the injections. The main figure shows the pattern of the tumor growth over 7 weeks of injections of liposomes (LIP) containing doxorubicin (DXR) at the indicated concentrations or liposomes without drug (LIP without DXR). The results demonstrate that the targeted liposomes are the most efficient method for minimizing tumor growth. The tumor volume is plotted as a mean and standard error. The error bars on the LIP (DXR) 15 mg/kg group are contained within the symbol for this group.

FIG. 10 is a graph showing results obtained in vivo with siRNA H-Ferritin. For this study, a subcutaneous tumor model was used to show the in vivo efficacy of the siRNA H-ferritin approach. The siRNA for H-ferritin or the nonsense (NS) control was first conjugated into liposomes and then injected directly into a subcutaneous glioblastoma tumor growing in the flank of nude mice. The concentration of siRNA or NS RNA injected into the tumor was ˜4 μg. After injection of the siRNA, the mice, received 25 μM of BCNU delivered i.p. 24 hours. The injections were performed once a week. As can be seen in this figure, the rate of tumor shrinkage was significantly faster in the animals receiving siRNA in the tumors as opposed to NS RNA. The significance of the data in this graph are two-fold: 1) the data provide proof of concept that siRNA for H-ferritin delivered into tumors will enhance the efficacy of standard chemotherapeutic agents, 2) the siRNA can be delivered to the tumors using a liposome delivery system.

FIG. 11A is a scan of photographs showing images of a tumor (bright spot indicated by the arrow) in a rat 3 weeks after surgery to implant the tumor cells. The animal has not received any treatments. FIG. 11B is a scan of photographs showing the effect of treatment with Il-13 conjugated liposomes delivering doxorubicin. The liposomes were delivered by intravenous (tail vein) injection. The top 4 panels are images from the same rat in FIG. 11A after 2 injections over 3 weeks of IL-13 conjugated liposomes delivering doxorubicin (15 mg/kg). The bottom 2 images are also from the same rat after a third injection and 5 weeks post treatment. The arrow indicates the location of where the tumor had been. These results show that an intravenous approach to deliver nanovesicles can be used to destroy brain tumors.

FIG. 12 is a schematic representation showing position 13 and 105 of interleukin-13 (IL-13) which are respectively glutamic acid (E) and lysine (K) which are responsible tumor associated receptor binding sites.

FIG. 13 is a schematic representation showing a preferred conjugation method.

FIG. 14 is a graph showing a cytotoxicity assay of IL13 and transferrin conjugated liposomes carrying doxorubicin on U251 cells. The cytotoxic potential of ligand targeted liposomes is, in general, higher at each concentration of DXR than the unconjugated liposomes carrying the same amount of doxorubicin. The presence of transferrin does not effect the toxicity of the liposomes. Statistical significance was determined by ANOVA* p<0.05, **p<0.01, *** p<0.001.

### DETAILED DESCRIPTION

A cargo moiety may be associated with a particle in any of various ways. In one embodiment, a cargo moiety is bonded to a particle, for example by a covalent bond. In another embodiment, a cargo moiety is encapsulated in a particle.

### Definitions

The term “specific binding” refers to that binding which occurs between such paired species as enzyme/substrate, receptor/agonist, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody preferably binds to a single epitope and to no other epitope within the family of proteins.

The terms “ligand” or “targeting moiety”, as used herein, refer generally to all molecules capable of specifically binding to a particular target molecule and forming a bound complex as described above. Thus the ligand and its corresponding target molecule form a specific binding pair. Examples include, but are not limited to antibodies, lymphokines, cytokines, receptor proteins such as CD4 and CD8, solubilized receptor proteins such as soluble CD4, hormones, growth factors, and the like which specifically bind desired target cells, and nucleic acids which bind corresponding nucleic acids through base pair complementarity. Other preferred targeting moieties include antibodies and antibody fragments (e.g., the Fab′ fragment).

As used herein, “cancer” refers to all types of cancer or neoplasm or malignant tumors found in mammals, including, but not limited to: leukemias, lymphomas, melanomas, carcinomas and sarcomas. Examples of cancers are cancer of the brain, breast, pancreas, cervix, colon, head & neck, kidney, lung, non-small cell lung, melanoma, mesothelioma, ovary, sarcoma, stomach, uterus and Medulloblastoma. The term “cancer” includes any cancer arising from a variety of chemical, physical, infectious organism cancer causing agents. For example, hepatitis B virus, hepatitis C virus, human papillomaviruses; sun; lead and lead compounds, X-rays, compounds found in grilled meats, and a host of substances used in textile dyes, paints and inks. Further details of cancer causing agents are listed in The Report on Carcinogens, Eleventh Edition. Federal law requires the Secretary of the Department of Health and Human Services to publish the report every two years.

Additional cancers which can be treated by the disclosed composition according to the invention include but not limited to, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, and prostate cancer.

“Diagnostic” or “diagnosed” means identifying the presence or nature of a pathologic condition or a patient susceptible to a disease. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The terms “patient” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. In tumor (e.g., cancer) treatment, the composition can directly decrease the pathology of tumor cells, or render the tumor cells more susceptible to treatment by other therapeutic agents, e.g., radiation and/or chemotherapy.

The treatment of neoplastic disease, cancer, or neoplastic cells, refers to an amount of the composition, described throughout the specification and in the Examples which follow, capable of invoking one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention of tumor cell infiltration into peripheral organs; (6) inhibition, including (i) reduction, (ii) slowing down or (iii) complete prevention of metastasis; (7) enhancement of anti-tumor immune response, which may result in (i) maintaining tumor size, (ii) reducing tumor size, (iii) slowing the growth of a tumor, (iv) reducing, slowing or preventing invasion or (v) reducing, slowing or preventing metastasis; and/or (8) relief, to some extent, of one or more symptoms associated with the disorder.

The terms “dosing” and “treatment” as used herein refer to any process, action, application, therapy or the like, wherein a subject, particularly a human being, is rendered medical aid with the object of improving the subject's condition, either directly or indirectly.

The treatment of a patient compositions of the invention, can be combined with one or more therapies. For example, in the case of treating cancer, the patient may be treated with a combination of the targeting liposome carrying a cargo moiety and a regimen of chemotherapeutic agents. The cargo moiety can be any chemotherapeutic agent. A “chemotherapeutic agent” which can also be the cargo moiety for treatment of a tumor is a chemical compound useful in the treatment of cancer. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carnomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®, Bristol-Myers Squibb Oncology, Princeton, N.J.) and docetaxel (TAXOTERE®, Rhône-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

Treatment of an individual suffering from an infectious disease organism refers to a decrease and elimination of the disease organism from an individual. For example, a decrease of viral particles as measured by plaque forming units or other automated diagnostic methods such as ELISA etc.

### Compositions

A preferred particulate vehicle is a liposome. The term “liposome” or “nanovesicle” as used herein refers to a particle including lipid-containing molecules arranged to form a unilamellar or multilamellar membrane wall surrounding an interior volume. The interior volume may be aqueous. A cargo moiety may be encapsulated in the interior volume of a liposome for delivery to a target. Some cargo molecules may be bonded to an exterior surface of a membrane wall for delivery to a target. A liposome advantageously protects a cargo moiety from metabolic processes and exposure to denaturing environments during transport to a desired site of action.

The immunoliposomes in accordance with the present invention are also designed for delivering therapeutic genes across the blood-brain barrier followed by expression in the brain of the therapeutic agents encoded by the gene. However, these liposomes or complexes can be used for targeting and delivery of the cargo to any location in vivo. The liposomes are a form of nanocontainer and nanocontainers, such as nanoparticles or liposomes, are commonly used for encapsulation of drugs. A liposome vehicle included in a delivery system according to the present invention is formulated and sized to optimize crossing the blood brain barrier in order to deliver a cargo moiety to a CNS target.

In a particular formulation, a liposome vehicle has a diameter in the range of about 1-1000 nanometers. In a further embodiment, a liposome vehicle has a diameter in the range of about 10-250 nanometers. In a further preferred embodiment, a liposome vehicle has a diameter in the range of about 50-150 nanometers. Restricting the size of liposomes enhances the potential of the liposomes to cross the blood-brain barrier.

Liposomes included in a system according to the invention include lipids such as positively charged lipids, neutral lipids, negatively charged lipids, amphiphilic lipids and may include phospholipids, cholesterols, and stearylamines for example. General liposome compositions and methods for making them are described in references such as Liposomes: A Practical Approach, The Practical Approach Series, 264, V. P. Torchilin and V. Weissig (Eds.) Oxford University Press; 2nd ed., 2003. Suitable types of liposomes are made with neutral phospholipids such as 1-palmitoyl-2-oleoyl-sn-glycerol-3-phosphocholine (POPC), diphosphatidy phosphocholine, distearoylphosphatidylethanolamine (DSPE), or cholesterol, along with a small amount (1%) of cationic lipid, such as didodecyldimethylammonium bromide (DDAB) to stabilize the anionic DNA within the liposome.

Particular liposome formulations useful in an inventive system are described herein.

In particular embodiments of the present invention, a liposome component may be included to affect pharmacokinetics and biodelivery of the liposome vehicles and their cargo. For example, polyethylene glycol (PEG) not only aids in targeting the vehicle to a target, such as tumors, but also renders the liposomes unable to be cleared by the reticuloendothelial system and increases circulation half-life of the liposomes. Thus, in some embodiments, a PEG modified component is included in a liposome vehicle.

A cargo moiety delivered in association with a vehicle included in an inventive system may be any of various therapeutic and diagnostic agents which are desired to be delivered to a CNS target. Therapeutic agents which can be included as cargo moieties in the delivery system of the present invention illustratively include but are not limited to therapeutic compounds such as an analgesic, an anesthetic, an antibiotic, an anticonvulsant, an antidepressant, an antimicrobial, an anti-inflammatory, anti-migraine, an antineoplastic, an antiparasitic, an antitumor agent, an antiviral, an anxiolytic, a cytostatic, a hypnotic, a metastasis inhibitor, a sedative and a tranquilizer. Diagnostic agents that may be included in the delivery system of the present invention as cargo moieties illustratively include but are not limited to a contrast agent, a labeled imaging agent such as a radiolabeled imaging agent, and an antitumoral antibody. Combinations of therapeutic compounds may be included, combinations of diagnostic agents may be included, and combinations of both therapeutic and diagnostic agents may be included. Further suitable therapeutic and diagnostic compounds that may be delivered by a system according to the invention may be found in standard pharmaceutical references such as A, R. Gennaro, Remington: The Science and Practice of Pharmacy, Lippincott Williams & Wilkins, 20th ed. (2003); L. V. Allen, Jr. et al., Ansel's Pharmaceutical Dosage Forms and Drug Delivery Systems, 8th Ed. (Philadelphia, Pa.: Lippincott, Williams & Wilkins, 2004); J. 0. Hardman et al., Goodman & Gilmans The Pharmacological Basis of Therapeutics, McGraw-Hill Professional, 10th ed. (2001).

In one embodiment, a cargo moiety includes iron. Selective delivery of iron to the brain may be used to treat neurological conditions associated with brain iron deficiency. For example, ‘Restless Legs Syndrome” affects 10-15% of the adult population may be a target disorder for deli very of iron as a therapeutic agent. Further, developmental iron deficiency is considered by the World Health Organization to be the number one health problem and delivery of iron using a system according to the present invention may aid in treatment of this deficiency. Attention Deficit Disorder is another condition which may be ameliorated by delivery of iron to the brain. It is estimated that as many as 20% of individuals with Attention Deficit Disorder may have low brain iron levels. In addition, an iron containing cargo moiety may be delivered as a diagnostic agent to assist in imaging tumors and/or neuritic plaques in the brain. Iron is a contrast enhancer and selectively targeting iron loaded nanoparticles to tumors and/or plaques may aid in imaging techniques such as MRI.

In a preferred embodiment, a cargo moiety is an anti-cancer compound which inhibits or prevents abnormal cell growth and/or which destroys or damages an abnormal cell. In particular embodiments, an anti-cancer composition included as a cargo moiety is an anti-tumoral compound. Also preferred are embodiments including an anti-cancer composition which is an antineoplastic agent, a cytostatic agent, and/or a metastasis inhibitor. An anti-cancer cargo moiety may be in any of various forms such as a nucleic acid, oligonucleotide, protein, peptide, and/or chemical compound.

In a further preferred embodiment, a cargo moiety is an siRNA composition, the siRNA directed at a target to be regulated. For example, siRNA directed towards down-regulation of ferritin in a cancer cell is included as a cargo moiety.

As noted above, CNS therapeutic and diagnostic targets are problematic due to the complexity of the CNS which includes many cell types. In cases where a particular discrete region of the brain is to be treated, it is often difficult to isolate the targeted cells from those in the vicinity. For example, in most organs afflicted with cancer, the surgeon removes all vestiges of visible tumor plus a generous amount of surrounding tumor in attempt to prevent recurrence. Malignant brain tumors pose a unique dilemma in regard to resection. As is evident in high grade astrocytomas, local infiltration prevents the complete resection of all malignant cells. Wide tumor margins are not attainable due to the potential post-surgical damage that will ensue. It is therefore critical to develop targeted delivery systems that cross the blood brain barrier and ablate individual cancer cells without causing diffuse damage to surrounding brain tissue. Additionally, use of targeted delivery vehicles for therapeutic and diagnostic agents to treat brain tumors might obviate the need for anesthesia and/or lumbar puncture in patients. Thus, delivery systems and methods are required which are capable of delivering a variety of anticancer agents to brain tumors in a manner that increases tumor accumulation, increases the indices of therapeutic agents and decreases the toxic side effects to normal cells.

Targeting Moieties: An optional targeting moiety is associated with a delivery vehicle in order to specifically target the delivery vehicle to a particular cell type. In a particular option, the targeting moiety specifically binds to a receptor on a particular cell type.

In an example of a specific type of CNS tumor, high-grade astrocytomas are completely inaccessible to surgery because of the essential surrounding tissues that may be harmed during surgery. Radiation and high-dose chemotherapy have both been shown to cause extensive, life-altering side effects with questionable gain. It is, therefore, critical to develop targeted delivery systems that ablate individual cancer cells without causing diffuse damage to surrounding brain tissue. To do this, these delivery systems need to be able to specifically target the astrocytoma and be able to traverse the blood-brain barrier.

Human IL-13 is a cytokine secreted by activated T cells that elicits both pro-inflammatory and anti-inflammatory immune responses (McKenzie, A. N., et al. (1993) PNAS USA 90, 3735-3739; Minty, A., et al. (1993) Nature 362, 248-250). IL-13 has two types of receptors: IL-13/4R is present on normal cells and binding is shared with IL-4, while IL-13Rα2 does not bind IL-4 and is expressed primarily in malignancy (Caput, D., et al. (1996) Journal of Biological Chemistry 271, 16921-16926). High-grade astrocytomas and pilocytic astrocytomas are reported to overexpress the brain tumor specific IL-13Rα2 receptor (Debinski, W., (2000) J. Neuro-Oncology 48, 103-111; Kawakami, M. et al. (2004) Cancer 101: 1036-1042). These malignant brain tumors are heterogeneous, rapidly progressive and extremely resistant to current therapies. High-grade astrocytomas (HGA) are considered the most devastating brain tumors due to their rapid and infiltrative growth and the overall poor prognosis of patients with the disease. HGAs, which include glioblastoma multiforme (GBM), are rapidly progressive heterogeneous brain tumors of glial origin that are extremely resistant to current therapies.

IL-13Rα2 is associated with high grade astrocytomas (HGA) and is not significantly expressed in normal tissue with the exception of the testes (Caput, D., (1996) J. Biological Chemistry 271, 16921-16926; Debinski, W. et al. (2000) J. Neuro-Oncology 48, 103-111; Debinski, W. et al. (2000) Mol. Med. 6, 440-449). A recent study determined that pilocytic astrocytomas, the most common astrocytic tumors in children, also overexpress the IL-13Rα2 receptor (Kawakami, M. et al. (2004) Cancer, 101, 1036-1042). These tumors account for 80-85% of cerebellar astrocytomas and 60% of optic gliomas (Campbell, J. W. et al (1996) J. Neuro-Oncology 28, 223-231; Alshail, B. et al. (1997) Brain Pathology 7: 799-806). Thus, the IL-13Rα2 receptor is an excellent target for delivering an anti-cancer cargo moiety, such as cytotoxic agents, to a variety of devastating brain tumors.

An inventive liposome based molecular delivery system is provided which is capable of delivering a cargo moiety, such as toxic, immune-stimulating or genetic material, to a tumor cell in the CNS. In particular, a delivery system according to the present invention increases efficacy of the delivered cytotoxic agents and decreases toxicity to normal cells.

Thus, in one embodiment of the present invention, a ligand for an IL-13 receptor expressed by a CNS tumor cell is a targeting moiety which is associated with a particulate delivery vehicle in order to target a therapeutic and/or diagnostic cargo moiety carried by the vehicle to a cell expressing the IL-13 receptor.

An IL-13 receptor targeting moiety includes wild-type IL-13 and mutants of IL-13 which have a higher binding affinity for an IL-13 receptor than the wild-type IL-13. In a further embodiment, an IL-13 receptor targeting moiety includes mutants of IL-13 which have a higher binding affinity for an IL-13Rα2 receptor than the wild-type IL-13.

Mutants of IL-13 that are superagonistic towards GBM associated IL-13Rα2 are used to target liposomes carrying cytotoxic agents to brain tumors in methods according to the present invention. Particular compositions and methods of the present invention target this glioma specific receptor using IL-13, its high affinity mutants, IL13.K105R (Madhankumar, A. B. et al. (2004) Neoplasia (New York) 6, 15-22) and IL13.E13K, another mutant that is more specific and has enhanced avidity towards the cancer associated receptor IL-13Rα2 (Debinski, W., et al. (1998) Nature Biotechnology 16, 449-453). A delivery vehicle conjugated to IL-13 and/or its high affinity mutants delivers chemotherapeutic agents specifically to brain tumors without affecting normal, healthy tissues.

The following example is not to be construed as a limitation of the invention. When IL-13 is chemically conjugated to the surface of the liposomes, as described infra, these liposomes specifically target high grade astrocytomas (HGA) without affecting the normal brain tissue. HGAs are a highly aggressive malignant brain tumor and are always fatal. These ligand targeted liposomes carrying the chemotherapeutic agent can cross the blood brain barrier, without release of their contents and are thus, suitable for intravenous or intraperitoneal delivery as well as traditional methods involving intratumoral injection. We also describe the specific binding and internalization of the targeted liposomes. Furthermore, we have established that the targeted liposomes can encapsulate cytotoxins, engineered gene products or contrast enhancement agents in an enhanced and specific mode. We have verified the cytotoxic behavior of DXR encapsulated liposomes on glioma cells and have shown that the liposomes can deliver engineered genes. A particular advantage of our delivery system is the avoidance of multidrug resistance (MDR), which results in decreased accumulation of the drugs in most of the cancer cells and in vivo tumors, and expulsion by the blood-brain barrier resulting in increased drug efflux and decreased efficiency of the cytotoxic agent Thus, the IL-13 receptor targeted nanovesicles encapsulated with therapeutic agents that require specific delivery to tumor cells. These nanovesicles transcytose the BBB and circumvent the MDR efflux mechanism.

Compositions and methods according to the present invention targeting high-grade and certain low-grade astrocytomas or other cells that overexpress the cancer associated receptor for interleukin-13, IL-13Rα2 have advantages over conventional chemotherapies that possess serious drawbacks, like difficulties with multi-drug resistance and P-glycoprotein mediated drug efflux, resulting in poor delivery through the blood-brain or blood-tumor barrier. Transport of therapeutic and diagnostic agents across the blood-brain barrier and targeting specific receptors allows administration of these therapeutic drugs and diagnostic agents through an intravenous route, an advantage in brain cancer therapy.

A second targeting moiety may be associated with a delivery vehicle for use in an inventive method and/or inventive compositions in addition to or instead of IL-13 and/or IL-13 mutants. For example, transferrin is optionally included as a targeting molecule which may aid in transport of a delivery vehicle across the blood brain barrier (BBB). Additional targeting moieties include a second receptor ligand, an antibody, a lectin, a carbohydrate, an enzyme, an enzyme substrate, or a fragment of any of these sufficient to specifically interact with a target cell.

Method of conjugation: We have shown results (see, for example, the Examples which follow) in the in vitro targeting experiments with the liposomes conjugated by the method mentioned infra, we are also conjugating by another preferred method in which we are including the lipids DSPE-PEG maleimide or MCC-PE in the liposome composition which are available as such from Avanti polar lipids. By this way the thio containing proteins and peptides can be conjugated to the liposomes. This method is particularly preferred for in vivo use since MCC and other maleimide forms more stable complexes that can survive in serum longer(1) and MCC contains more stable maleimide function group towards hydrolysis in aqueous reaction environments (Hashida, S., and Ishikowa, E. (1985). Use of normal IgG and its fragments to lower the non-specific binding of Fab5-enzyme conjugates in sandwich enzymes immunoassay. Anal. Lett. 18(b9), 1143-1155; Dewey, R. E. et al. (1987) Proc. Natl. Acad. Sci. U.S.A. 84, 5374-5378).

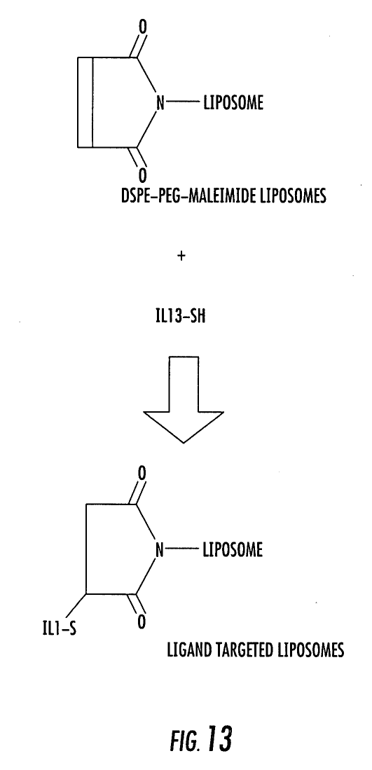

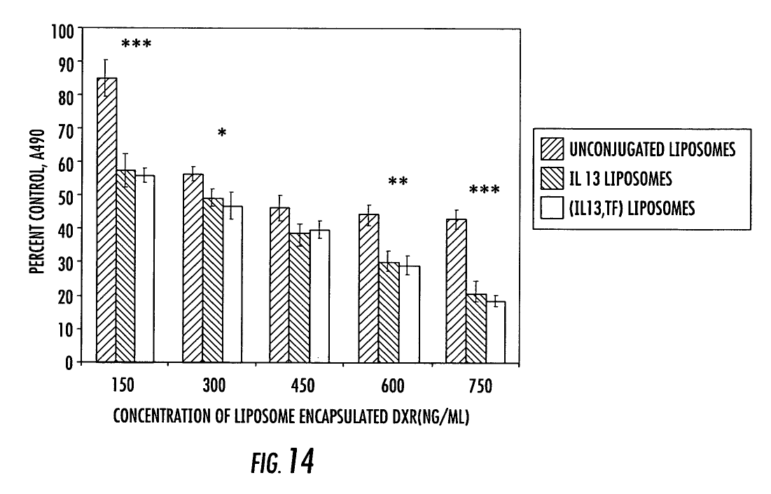

Thus for example we are using the same composition of lipids for making the liposomes as discussed in the examples which follow, with an additional lipid of MCC (1,2-Dipalmitoyl-sn-glycero-3-phosphoethanolamine-N-[4-(p-maleimidomethyl)cyclohexane-carboxamide]) or DSPE-PEG-Maleimide (1,2-Distearoyl-sn-Glycero-3-Phosphoethanolamine-N-[Maleimide(Polyethylene Glycol)2000] (Ammonium Salt)) (0.5 mol). Here we thiolate the IL-13 protein by treating the protein with imminothiolane (Traut's reagent) and then the unmodified excess imminothiolane was removed by passing through Spehadex G25M column. This thiolated IL-13 was directly added to IL-13 protein in pH 7.4 buffer and stirred for 1 h in the room temperature in nitrogen atmosphere. This was subsequently passed through Sepharose CL-2B column to purify or alternatively centrifuged at 40000 rpm to remove unconjugated protein. A schematic representation of the conjugation method is shown in FIG. 13.

Pharmaceutical composition: A pharmaceutical composition is provided according to the present invention which includes a plurality of particulate delivery vehicles, each particulate delivery vehicle having a wall defining an external surface and an internal volume, each particulate delivery vehicle having a cargo moiety associated therewith and, optionally, each particulate delivery vehicle having a targeting moiety associated therewith.

In a specific configuration, pharmaceutical composition includes a plurality of liposomes, each liposome having a wall defining an external surface and an internal volume, and each liposome having a cargo moiety associated therewith. In a preferred option, an IL-13 targeting moiety is associated with the liposomes so that they are targeted to a cell having an IL-13 receptor. Further preferred is inclusion of an IL-13 mutant, particularly an IL13.K105R and/or an IL13.E13K mutant, as a targeting moiety.

In general, a pharmaceutical composition will also include a pharmaceutically acceptable carrier to aid in administration of the plurality of particulate delivery vehicles. A pharmaceutically acceptable carrier is one which is essentially non-toxic to an individual to whom the composition is administered and which does not interfere with the integrity, bioavailability, or stability of the plurality of particulate delivery vehicles, the cargo moieties or the targeting moieties. The identity of a suitable carrier may be determined by the route of administration and the dosage form.

A method of treatment of an individual with a pharmaceutical composition according to the present invention includes administering a therapeutically effective amount of the composition. A therapeutically effective amount is that amount which achieves a therapeutic effect without substantial undesired side effects. Determination of an effective amount is within the usual practice of one of skill in the art and may be determined without undue experimentation.

As noted above, the blood-brain barrier (BBB) represents a formidable obstacle for delivering therapeutic and/or diagnostic agents to the CNS. A method according to the present invention includes use of a cell culture model of the blood brain barrier. Such a method allows for testing the ability of IL-13 and mutants thereof conjugated to a delivery vehicle, such as liposomes, to traverse the BBB while maintaining relative selectivity for tumor cells. Thus, systems and methods are provided according to the present invention for directly evaluating and optimizing transport of vehicles such as nanovesicles and liposomes into the brain. Also provided is a system for evaluating BBB transport of delivery vehicles for targeted cargo delivery includes an endothelial cell culture system. In a particular embodiment, non-human endothelial cells such as bovine retinal endothelial cells and rat brain endothelial cells are used. A provided system and methods of use allow for rapid evaluation of drug delivery systems and a low cost evaluative system for modifications to any drug delivery system.

A method of determining the extent of BBB transport of a substance includes providing a BBB model system which includes a first reservoir, a second reservoir, and a cellular transport inhibitor extending between the first and second reservoirs, inhibiting transport of specified substances between the first and second reservoirs, the specified substances being those which do not pass the in vivo blood brain barrier. A medium is present in the first and second reservoirs. A control sample of medium is taken from the second reservoir prior to testing to establish a baseline. A query substance is added to the first reservoir and samples of a medium present in the second reservoir are taken at intervals over a period of time and tested for presence of the query substance and/or metabolites thereof. The ability of the query substance to pass through the cellular transport inhibitor is compared to the ability of a substance characterized with respect to its ability to pass through the BBB in vivo. A cellular transport inhibitor includes endothelial cells capable of forming tight junctions in vitro. A BBB model system and methods of use thereof are optimized for testing of the ability of liposomal compositions to pass through the BBB in one embodiment.

It is appreciated that while the present specification details methods and compositions pertaining to human IL-13 and mutants thereof, animal IL-13 and mutants thereof may bind to receptors described herein to provide the targeting function required.

### Nucleic Acids

In a preferred embodiment of the invention, the liposomes formed of the lipids described above are associated with a nucleic acid. By “associated” it is meant that a therapeutic agent, such as a nucleic acid, is entrapped in the liposomes central compartment and/or lipid bilayer spaces, is associated with the external liposome surface, or is both entrapped internally and externally associated with the liposomes. It will be appreciated that the therapeutic agent can be a nucleic acid or a drug compound. I t will also be appreciated that a drug compound can be entrapped in the liposomes and a nucleic acid externally associated with the liposomes, or vice versa.

In a preferred embodiment of the invention, a nucleic acid is associated with the liposomes. The nucleic acid can be selected from a variety of DNA and RNA based nucleic acids, including fragments and analogues of these. A variety of genes for treatment of various conditions have been described, and coding sequences for specific genes of interest can be retrieved from DNA sequence databanks, such as GenBank or EMBL. For example, polynucleotides for treatment of viral, malignant and inflammatory diseases and conditions, such as, cystic fibrosis, adenosine deaminase deficiency and AIDS, have been described. Treatment of cancers by administration of tumor suppressor genes, such as APC, DPC4, NF-1, NF-2, MTS1, RB, p53, WT1, BRCA1, BRCA2 and VHL, are contemplated.

Examples of specific nucleic acids for treatment of an indicated conditions include: HLA-B7, tumors, colorectal carcinoma, melanoma; IL-2, cancers, especially breast cancer, lung cancer, and tumors; IL-4, cancer; TNF, cancer; IGF-1 antisense, brain tumors; IFN, neuroblastoma; GM-CSF, renal cell carcinoma; MDR-1, cancer, especially advanced cancer, breast and ovarian cancers; and HSV thymidine kinase, brain tumors, head and neck tumors, mesothelioma, ovarian cancer.

The polynucleotide can be an antisense DNA oligonucleotide composed of sequences complementary to its target, usually a messenger RNA (mRNA) or an mRNA precursor. The mRNA contains genetic information in the functional, or sense, orientation and binding of the antisense oligonucleotide inactivates the intended mRNA and prevents its translation into protein. Such antisense molecules are determined based on biochemical experiments showing that proteins are translated from specific RNAs and once the sequence of the RNA is known, an antisense molecule that will bind to it through complementary Watson-Crick base pairs can be designed. Such antisense molecules typically contain between 10-30 base pairs, more preferably between 10-25, and most preferably between 15-20.

The antisense oligonucleotide can be modified for improved resistance to nuclease hydrolysis, and such analogues include phosphorothioate, methylphosphonate, phosphodiester and p-ethoxy oligonucleotides (WO 97/07784).

The entrapped agent can also be a ribozyme, DNAzyme, or catalytic RNA.

The nucleic acid or gene can, in another embodiment, be inserted into a plasmid, preferably one that is a circularized or closed double-stranded molecule having sizes preferably in the 5-40 Kbp (kilo basepair) range. Such plasmids are constructed according to well-known methods and include a therapeutic gene, i.e., the gene to be expressed in gene therapy, under the control of suitable promoter and enhancer, and other elements necessary for replication within the host cell and/or integration into the host-cell genome. Methods for preparing plasmids useful for gene therapy are widely known and referenced.

Polynucleotides, oligonucleotides, other nucleic acids, such as a DNA plasmid, can be entrapped in the liposome by passive entrapment during hydration of the lipid film. Other procedures for entrapping polynucleotides include condensing the nucleic acid in single-molecule form, where the nucleic acid is suspended in an aqueous medium containing protamine sulfate, spermine, spermidine, histone, lysine, mixtures thereof, or other suitable polycationic condensing agent, under conditions effective to condense the nucleic acid into small particles. The solution of condensed nucleic acid molecules is used to rehydrate a dried lipid film to form liposomes with the condensed nucleic acid in entrapped form.

The therapeutic gene can also be encapsulated (a cargo moiety) within the liposome can be any of the common therapeutic genes which are used to express therapeutic and diagnostic agents. Exemplary therapeutic genes include brain-derived neurotrophic factor (BDNF) for treatment of neurodegenerative disease, stroke, or brain trauma; tyrosine hydroxylase and/or aromatic amino acid decarboxylase for Parkinson's disease; β-glucuronidase; hexosaminidase A; herpes simplex virus thymidine kinase or genes encoding antisense RNA to the epidermal growth factor receptor for treatment of brain tumors; lysosomal storage disorder replacement enzymes for Tay-Sachs and other lysosomal storage disorders; gene encoding antisense RNA for the treatment of the cerebral component of acquired immune deficiency syndrome (AIDS). In addition to the therapeutic gene, the plasmid DNA may also contain DNA sequences either before or after the therapeutic sequence and these additional parts of the plasmid may promote tissue-specific transcription of the plasmid in a particular cell in the brain, may promote enhanced translation and/or stabilization of the mRNA of the therapeutic gene, and may enable episomal replication of the transgene in brain cells. In general, the therapeutic gene will contain at least 100 nucleotides or have a molecular weight above 30,000 Daltons. It is preferred that the therapeutic gene be contained within a plasmid or other suitable carrier for encapsulation within the internal compartment of the liposome or nanocontainer.

The therapeutic gene may be encapsulated within the liposome according to any of the well known drug encapsulation processes. For example, encapsulation by sonication, freeze/thaw, evaporation, and extrusion through membrane filters.

The number of therapeutic genes encapsulated within the liposome may vary from 1 to many, depending on the disease being treated. The limiting factor will be the diameter of therapeutic gene that is encapsulated within the liposome. Using polycationic proteins such as histone, protamine, or polylysine, it is possible to compact the size of plasmid DNA that contains several thousand nucleotides to a structure that has a diameter of 10-30 nm. The volume of a 100 diameter liposome is 1000-fold and 35-fold greater than the volume of a 10 nm and 30 nm DNA compacted sphere, respectively. Therefore, it is possible to encapsulate many copies of the same gene or multiple copies of multiple genes within the liposome.

### Other Targeting Ligands

The liposomes may optionally be prepared to contain surface groups, such as antibodies or antibody fragments, small effector molecules for interacting with cell-surface receptors, antigens, and other like compounds, for achieving desired target-binding properties to specific cell populations. Such ligands can be included in the liposomes by including in the liposomal lipids a lipid derivatized with the targeting molecule, or a lipid having a polar-head chemical group that can be derivatized with the targeting molecule in preformed liposomes. Alternatively, a targeting moiety can be inserted into preformed liposomes by incubating the preformed liposomes with a ligand-polymer-lipid conjugate.

Lipids can be derivatized with the targeting ligand by covalently attaching the ligand to the free distal end of a hydrophilic polymer chain, which is attached at its proximal end to a vesicle-forming lipid. There are a wide variety of techniques for attaching a selected hydrophilic polymer to a selected lipid and activating the free, unattached end of the polymer for reaction with a selected ligand, and in particular, the hydrophilic polymer polyethyleneglycol (PEG) has been widely studied (Allen, T. M., et al., Biochemicia et Biophysica Acta 1237:99-108 (1995); Zalipsky, S., Bioconjugate Chem., 4(4):296-299 (1993); Zalipsky, S., et al., FEBS Lett. 353:71-74 (1994); Zalipsky, S., et al., Bioconjugate Chemistry, 705-708 (1995); Zalipsky, S., in Stealth Liposomes (D. Lasic and F. Martin, Eds.) Chapter 9, CRC Press, Boca Raton, Fla. (1995)).

Targeting ligands are well known to those of skill in the art, and in a preferred embodiment of the present invention, the ligand is one that has binding affinity to endothelial tumor cells, and which is, more preferably, internalized by the cells. Such ligands often bind to an extracellular domain of a growth factor receptor. Exemplary receptors include the c-erbB-2 protein product of the HER2/neu oncogene, epidermal growth factor (EGF) receptor, basic fibroblast growth receptor (basic FGF) receptor and vascular endothelial growth factor receptor, E-, L- and P-selectin receptors, folate receptor, CD4 receptor, CD19 receptor, α,β-integrin receptors and chemokine receptors.

In other preferred embodiments, the liposome complexes may also be conjugated to transporter proteins to increase the transportation of the liposome complexes across membranes e.g. blood brain barrier, intestines, etc.

For example, in order to provide transport of the encapsulated therapeutic gene across the blood-brain barrier, a number of blood-brain targeting agents are conjugated to the surface of the liposome. Suitable targeting agents include insulin, transferrin, insulin-like growth factor, or leptin, as these peptides all have endogenous RMT systems within the BBB that also exist on the BCM, and these endogenous peptides could be used as “transportable peptides.” Alternatively, the surface of the liposome could be conjugated with 2 different “transportable peptides,” one peptide targeting an endogenous BBB receptor and the other targeting an endogenous BCM peptide. The latter could be specific for particular cells within the brain, such as neurons, glial cells, pericytes, smooth muscle cells, or microglia. Targeting peptides may be endogenous peptide ligands of the receptors, analogues of the endogenous ligand, or peptidomimetic MAbs that bind the same receptor of the endogenous ligand. The use of transferrin receptor (TfR)-specific peptidomimetic monoclonal antibodies as BBB “transportable peptides” are described in detail in U.S. Pat. Nos. 5,154,924; 5,182,107; 5,527,527; 5,672,683; 5,833,988; and 5,977,307. The use of an MAb to the human insulin receptor (HIR) as a BBB “transportable peptide” has been described (Pardridge, W. M., et al. (1995) Pharm Res., 12, 807-816).

The conjugation agents which are used to conjugate the blood-barrier targeting agents to the surface of the liposome can be any of the well-known polymeric conjugation agents such as sphingomyelin, polyethylene glycol (PEG) or other organic polymers. PEG is an especially preferred conjugation agent. The molecular weight of the conjugation agent is preferably between 1000 and 50,000 DA. A particularly preferred conjugation agent is a bifunctional 2000 DA PEG which contains a lipid at one end and a maleimide group at the other end. The lipid end of the PEG binds to the surface of the liposome with the maleimide group bonding to the receptor-specific monoclonal antibody or other blood-brain barrier targeting vehicle. It is preferred that from 5 to 1000 targeting vehicles be conjugated to each liposome. Liposomes having approximately 25-40 targeting vehicles conjugated thereto are preferred.

Exemplary combinations of liposomes, conjugation agents and targeting agents are as follows:

A transportable peptide such as insulin or an HIRMAb is thiolated and conjugated to a maleimide group on the tip of a small fraction of the PEG strands; or, surface carboxyl groups on a transportable peptide such as transferrin or a TfRMAb are conjugated to a hydrazide (Hz) moiety on the tip of the PEG strand with a carboxyl activator group such as N-methyl-N′-3(dimethylaminopropyl)carbodiimide hydrochloride (EDAC); a transportable peptide is thiolated and conjugated via a disulfide linker to the liposome that has been reacted with N-succinimidyl 3-(2-pyridylthio)proprionate (SPDP); or a transportable peptide is conjugated to the surface of the liposome with avidin-biotin technology, e.g., the transportable peptide is mono-biotinylated and is bound to avidin or streptavidin (SA), which is attached to the surface of the PEG strand.

Although the invention has been described using liposomes as the preferred nanocontainer, it will be recognized by those skilled in the art that other nanocontainers may be used. For example, the liposome can be replaced with a nanoparticle or any other molecular nanocontainer with a diameter<200 nm that can encapsulate the DNA and protect the nucleic acid from nucleases while the formulation is still in the blood or in transit from the blood to the intracellular compartment of the target cell. Also, the PEG strands can be replaced with multiple other polymeric substances such as sphingomylein, which are attached to the surface of the liposome or nanocontainer and serve the dual purpose of providing a scaffold for conjugation of the “transportable peptide” and for delaying the removal of the formulation from blood and optimizing the plasma pharmacokinetics. Further, the present invention contemplates delivery of genes to any group of cells or organs which have specific target receptors

### Pharmaceutical Compositions

Pharmaceutical compositions comprising the compositions of the invention are prepared according to standard techniques and further comprise a pharmaceutically acceptable carrier. Generally, normal saline will be employed as the pharmaceutically acceptable carrier. Other suitable carriers include, e.g., water, buffered water, isotonic solution (e.g., dextrose), 0.4% saline, 0.3% glycine, and the like, including glycoproteins for enhanced stability, such as albumin, lipoprotein, globulin, etc. These compositions may be sterilized by conventional, well known sterilization techniques. The resulting aqueous solutions may be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, etc. Additionally, the liposome compositions of the invention can be suspended in suspensions which include lipid-protective agents which protect lipids against free-radical and lipid-peroxidative damages on storage. Lipophilic free-radical quenchers, such as alphatocopherol and water-soluble iron-specific chelators, such as ferrioxamine, are suitable.

The concentration of liposome compositions of the invention in the pharmaceutical formulations can vary widely, i.e., from less than about 0.05%, usually at or at least about 2-5% to as much as 10 to 30% by weight and will be selected primarily by fluid volumes, viscosities, etc., in accordance with the particular mode of administration selected. For example, the concentration may be increased to lower the fluid load associated with treatment. This may be particularly desirable in patients having atherosclerosis-associated congestive heart failure or severe hypertension. Alternatively, complexes composed of irritating lipids may be diluted to low concentrations to lessen inflammation at the site of administration. The amount of compositions administered will depend upon the particular Fab′ used, the disease state being treated, and the judgment of the clinician. Generally the amount of composition administered will be sufficient to deliver a therapeutically effective dose of the nucleic acid. The quantity of composition necessary to deliver a therapeutically effective dose can be determined by one skilled in the art. Typical dosages will generally be between about 0.01 and about 50 mg nucleic acid per kilogram of body weight, preferably between about 0.1 and about 10 mg nucleic acid/kg body weight, and most preferably between about 2.0 and about 5.0 mg nucleic acid/kg of body weight. For administration to mice, the dose is typically 50-100 μg per 20 g mouse.

### Kits

The present invention also provides for kits for preparing the above-described liposome complexes/compositions. Such kits can be prepared from readily available materials and reagents, as described above. For example, such kits can comprise any one or more of the following materials: liposomes, nucleic acid (condensed or uncondensed), hydrophilic polymers, hydrophilic polymers derivatized with targeting moieties such as Fab′ fragments, and instructions. A wide variety of kits and components can be prepared according to the present invention, depending upon the intended user of the kit and the particular needs of the user. For example, the kit may contain any one of a number of targeting moieties for targeting the complex to a specific cell type, as described above.

Instructional materials for preparation and use of the liposome complexes can be included. While the instructional materials typically comprise written or printed materials, they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

Embodiments of inventive compositions and methods are illustrated in the following examples. These examples are provided for illustrative purposes and are not considered limitations on the scope of inventive compositions and methods.

### EXAMPLES

### Example 1

### IL-13 Mutants IL13.K105R and IL13.E13K as Ligands for Targeting Brain Tumor Associated Receptor

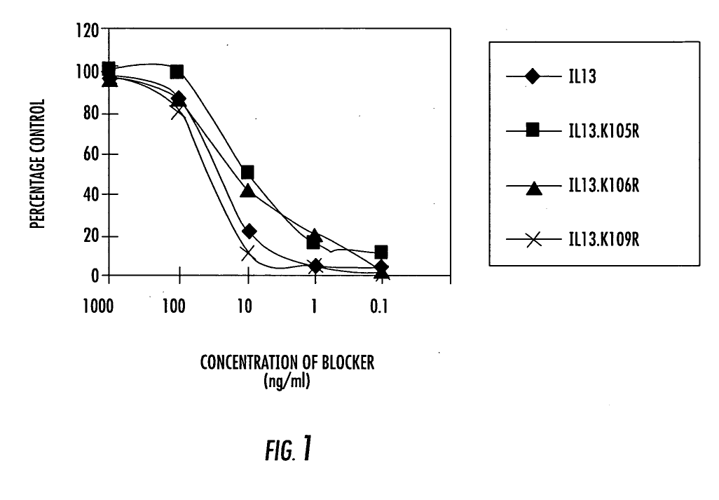

Through extensive alanine scanning mutagenesis of the D-helix region of IL-13 it is found that certain amino acids, like position K105 and R109, play an important role in binding the glioma associated receptor, IL13Rα2 (Madhankumar A. B. et al., (2002) J. Biological Chemistry 277, 43194-43205). Additionally, position K105 of this region of IL-13 is mutated into a variety of amino acids and the mutants' cytotoxic neutralization potential and binding affinity towards IL-13Rα2 is determined. FIG. 1 demonstrates the neutralization potential of the wild type IL-13 and its mutants at variable concentrations of the protein and at a fixed concentration of cytotoxin IL13-PE38QQR (10 ng/ml). In this titered cytotoxicity neutralization experiment, higher neutralization efficiency against the IL13-PE38QQR cytotoxin, for the mutant IL13.K105R is observed (Madhankumar A. B., Mintz, A., and Debinski, W. (2004) Neoplasia (New York) 6, 15-22). IL13.K105R is able to neutralize the cytotoxicity to 99% at 100 ng/ml concentration when compared with its blocking efficiency at 1000 ng/ml concentration. However, the wild type IL-13 and the mutant IL13.K106R and IL13.R109K have only 86.5%, 86.7% and 80.7% of the cytotoxicity neutralization efficiency with respect to their neutralization efficiency at 1000 ng/ml concentration. Thus, IL13.K105R has a high tumor associated receptor binding affinity and the mutant IL13.K105A has the least affinity as is evident from its cytotoxic neutralization potential. The high affinity mutant IL13.K105R is used as a ligand to conjugate to liposomes in order to better target the glioma associated IL-13Rα2 receptor. FIG. 1 shows results of a cytotoxicity neutralization assay with IL-13 and its mutants.

### Example 2

### Preparation and Characterization of IL-13 Conjugated Liposomes

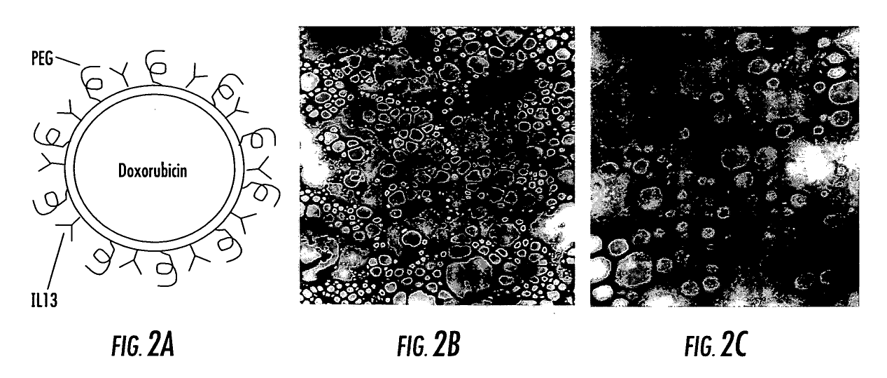

Sterically stable liposomes are formulated using distearophosphoethanolamine polyethyleneglycol 2000 (DSPE-PEG), dipalmitoylphosphatidylcholine (DPPC), cholesterol (CHOL), and stearylamine (SA) in a molar ratio of DPPC:CHOL:DSPE-PEO:SA=10:5:1.5:1.5. Liposomes are prepared by lipid film hydration followed by extrusion by a polycarbonate membrane extruder of gradually decreasing pore size to produce small unilamellar vesicles (SWV). The size distribution of the liposomes is determined by dynamic light scattering using a particle size analyzer, which is confirmed by Transmission Electron Microscope (TEM) using uranyl acetate as the staining agent. The average particle size is found to be 50-150 nm (FIG. 2B). This size is consistent with effective transport at the BBB.

FIG. 2(A) shows a schematic picture of PEG liposomes conjugated to the IL-13 molecule. FIG. 2(B) shows a TEM picture of IL-13-conjugated liposomes after staining with uranyl acetate (Particle size range=50-150 nm).

### Example 3

### Human IL-13 Conjugated Liposomes

To obtain human IL-13 conjugated liposomes (FIG. 1A), the gene for IL-13 from human testis is isolated using RT-PCR. IL-13 DNA is cloned into the TOPO-vector (Invitrogen), expressed in E. coli as His-tagged protein, and purified by nickel affinity binding. Mutations in the gene encoding for wild type IL-13 are introduced by unique site-elimination method in which site-specific mutations are introduced in the plasmid using a targeted mutagenic primer and a selection primer as suggested by the manufacturer. Primers are designed using Vector NTI Suite software (Bethesda, Md.). Conjugation of IL-13 and its mutants to liposomes is performed as follows: The heterobifunctional reagent SPDP (N-succimidyl-3(2-pyridyldithio)propionate) is employed to introduce pyridyl disulphide groups to the IL-13 molecule (Singh M. et al. (2001) European Journal of Pharmaceutics & Biopharmaceutics 52, 13-20). Briefly, 10 mol of SPDP is reacted with 1 mol of IL-13 in PBS for 24 hours followed by dialysis against PBS (MWCO 12-14000). The reaction mixture is reduced with DTT and isolated by gel filtration through a sephadex G2SM column. Thiolated IL-13 and liposomes are incubated overnight, and the conjugated liposomes are separated by ultracentrifugation.

### Example 4

### Binding and Internalization of IL-13 Conjugated Liposomes in Glioma Cells

Rhodamine labeled IL-13 liposomes are used to examine binding and internalization liposomes according to the invention. Such liposomes are incubated with U251 glioma cells and with normal glial cells as a control. There is relatively high binding and internalization of the IL-13 liposomes on glioma cells.

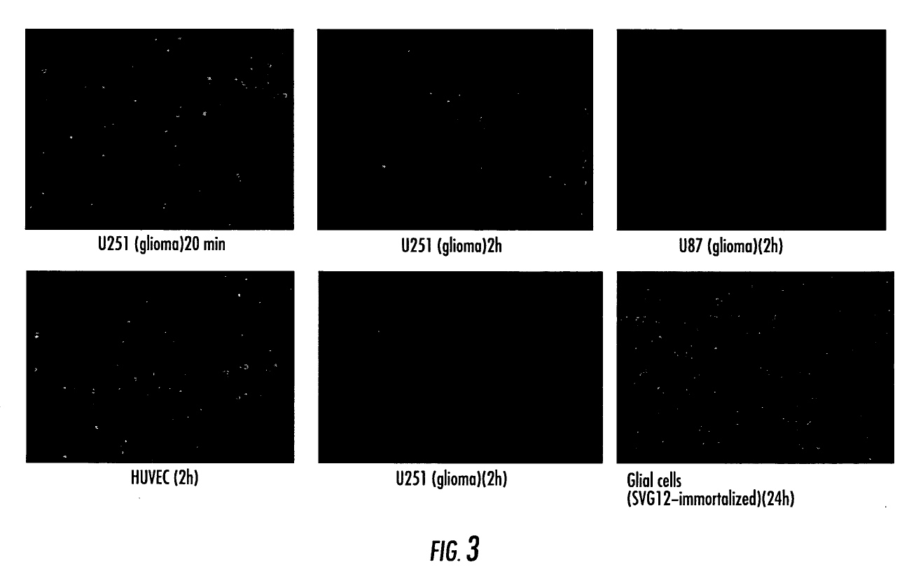

The liposomes have a weaker interaction with the normal glial cells and no internalization of the liposomes is detected as illustrated in FIG. 3. FIG. 3 shows binding and internalization of IL-13 conjugated rhodamine labeled liposomes on various glioma and normal cells.

IL-13 conjugated liposomes specifically target and become internalized into the glioma cells.

Internalization of IL13-liposomes on glioma cell lines: U251 glioma cells and normal glial cells were grown on chamber slides to subconfluency. Cells were then incubated with Rhodamine-PE labeled IL-13 liposomes for varying time periods. Then the cells were washed extensively with PBS, mounted and observed through a fluorescent microscope (Carl Zeiss, Inc., Germany).

Results and discussion: The use of SPDP as the conjugating agent to link IL13 to liposomes resulted in effective conjugation, which was verified by its immunoreactive against IL13 antibody on dotblot.

Fluorescence microscopy studies were performed to visualize the international and subsequent intracellular disposition of IL13 conjugated liposomes. Confocal microscopy of the rhodamine labeled IL13 ligand targeted liposome clearly showed the binding and international of IL13 conjugated ligand targeted liposomes on U251 glioma cell line. However, in the case of normal glial cells, although some non-specific binding was observed, internalization was not observed. These results clearly show the specificity of IL-13 conjugated liposomes on glioma cell lines.

The immunohistochemistry with rhodamine labeled IL-13-conjugated liposomes revealed the expression of IL13Rα2 receptor on most of the malignant tumors. Moreover, on the normal human cortex, the binding of liposomes was found to be least. This confirms that the IL13 conjugated liposomes will selectively bind the tumor and get internalized.

This study showed that those liposomes are more specific towards high grade and certain low grade tumors which express IL13R receptor.

### Example 5

### Targeting of Liposomes

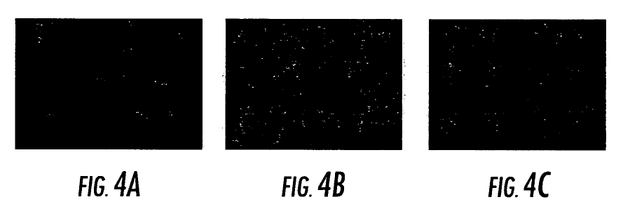

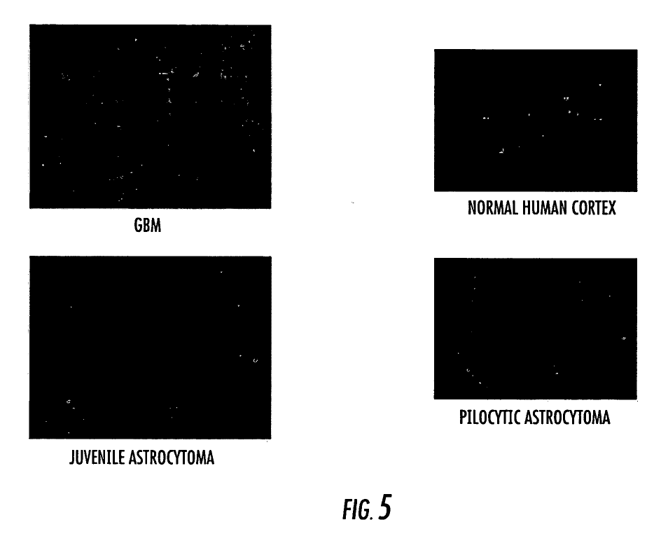

Targeting of inventive liposomes is performed using IL-13 conjugated liposomes, which are not labeled with rhodamine, and which contain doxorubicin (DXR) “encapsulated” in the liposomes. DXR has intrinsic fluorescence, and the fluorescence is detected in the glioma cells exposed to IL-13 conjugated liposomes (FIGS. 4A-4C) confirming the internalization of the liposomes and the ability of this nanovesicle system to delivery cytotoxins. FIGS. 4A-4C shows intrinsic fluorescence of doxorubicin (DXR) delivered to U251 glioma cells. DXR or liposomes carrying DXR at a concentration of 6 μg/ml are added to 5×10⁴ cells/well in a chamber slide and allowed to internalize for 24 hours before observing through fluorescence microscopy. FIG. 4A Free DXR; FIG. 4B DXR encapsulated in unconjugated liposomes, and FIG. 4C DXR encapsulated in IL-13 conjugated liposomes.

### Example 6

### Binding of Targets

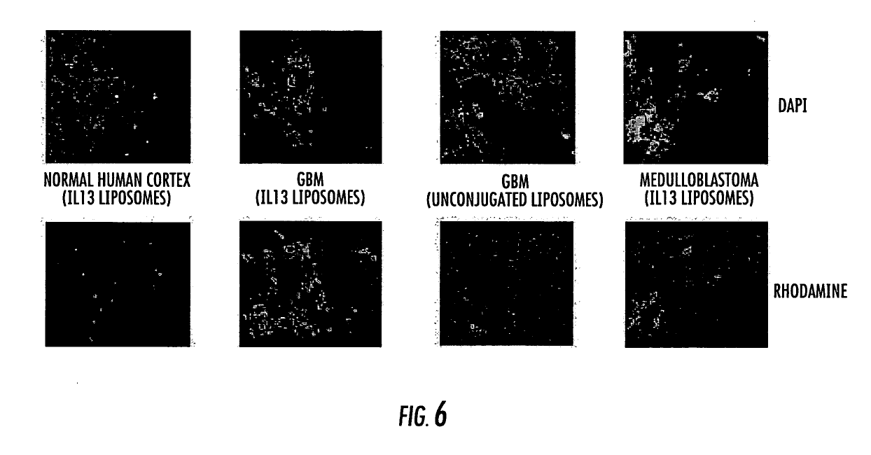

Glioblastoma multiforme (GBM) and pediatric brain tumor sections are treated with rhodamine labeled IL-13 liposomes and IL-13 liposomes encapsulating DXR after blocking nonspecific binding with 10% normal goat serum. There is a range of binding affinity of the IL-13 conjugated liposomes to the tumor sections as shown in FIGS. 5 and 6. Glioblastoma multiforme tumors show higher binding affinity towards IL-13 liposomes when compared with normal human brain sections (FIG. 5), which correlates to the level of IL-13Rα2 receptor expression. This is also supported by the internalization of DXR encapsulated IL-13 conjugated liposomes on GBM and on normal tumor sections as indicated by an intrinsic fluorescence of the DXR (FIG. 6). Thus, IL-13 conjugated liposomes can be utilized to target high-grade astrocytomas and low-grade pediatric brain tumors like juvenile astrocytoma.

FIG. 5 shows binding of rhodamine PE labeled IL-13 conjugated liposomes on various brain tumor sections and in normal brain indicating that higher specific binding is observed in Glioblastoma Multiforme (GBM), which overexpress IL-13Rα2 receptor. The binding pattern also shows that liposomes bind specifically to certain low grade astrocytomas. FIG. 6 shows intrinsic fluorescence of the DXR after exposing the IL-13 conjugated and unconjugated liposomes containing encapsulated DXR on tumor sections.

### Example 7

### Delivery of Cargo

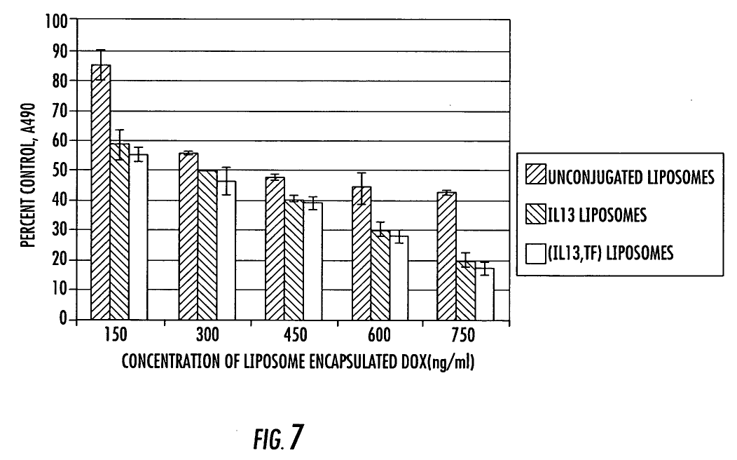

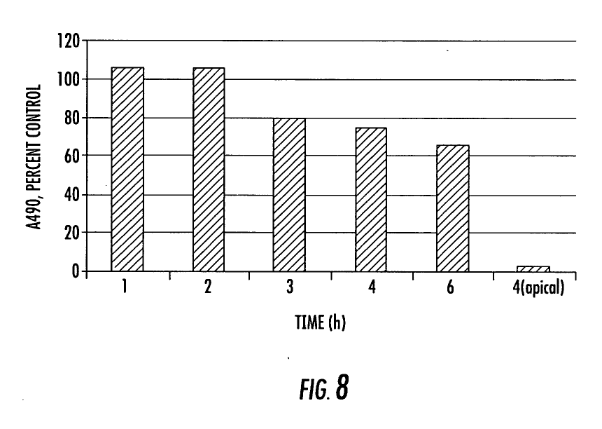

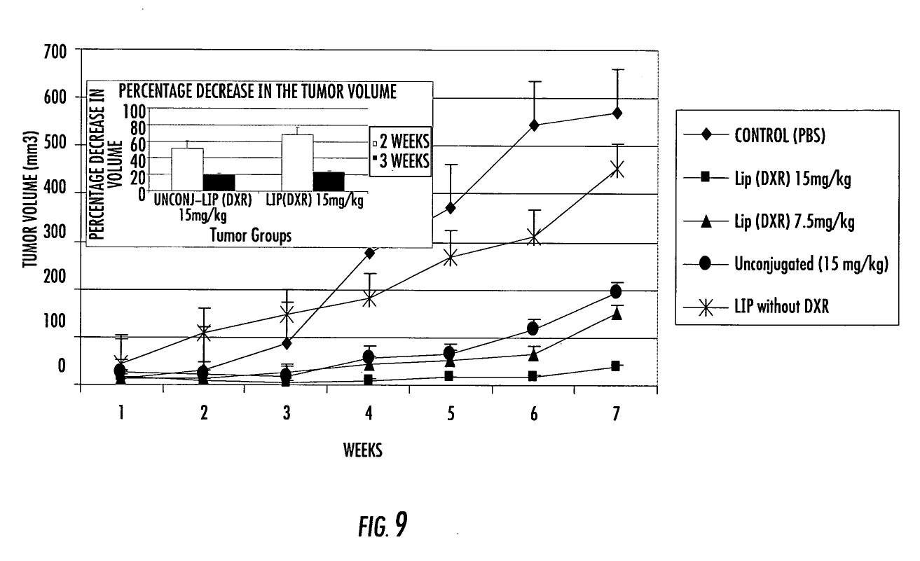

To demonstrate that the liposomes deliver toxic amounts of DXR, U251 glioma cells are treated with IL-13 liposomes encapsulating DXR. The liposomal delivered DXR is associated with enhanced cytotoxicity compared to non-conjugated liposomes as shown in FIG. 7. FIG. 7 illustrates a cytotoxicity assay of the IL-13 conjugated liposomes carrying DXR on U251 glioma cells. The cytotoxicity of IL-13 liposomes is higher than the unconjugated liposomes carrying the same amount of DXR. This observation demonstrates that receptor mediated endocytosis of IL-13 conjugated liposomes results in enhanced delivery of the encapsulated DXR resulting in higher cytotoxicity.

### Example 8

### Cytotoxicity Assays

Cytotoxicity experiments are performed with the media collected from the basal chamber of the EBB model described above. First, 2.5×10³ cells (U251 glioma cells) per well are plated in a 96 well plate in a total volume of 150 microliters. After a 24 hour incubation, 50 microliters of basal media or apical media from each collected time point is added to the cells and incubated for 48 hours. At the end of the incubation, the number of proliferating cells is measured by the colorimetric MTS/PMS assay (Promega, Madison, Wis.). Cells treated with BSA and cycloheximide serve as positive and negative controls for the cytotoxicity assay.

A cytotoxicity experiment is performed with the media from the basal chamber on U251 glioma cells and results show a clear decrease over time in the number of live cells (FIG. 8). FIG. 8 shows a bar graph representing cytotoxicity experiments performed with media from the BBB transport experiment. IL-13 conjugated liposomes with encapsulated DXR are added to the apical chamber of the BEE model (described above) and allowed to undergo transport for specific amounts of time as denoted. U251 glioma cells are treated with media collected from the basal chamber. Over time the basal media becomes more cytotoxic demonstrating that the targeted liposomes can traverse the BREC layer of cells in our BBB model. Also shown is the cytotoxicity of the apical media after 4 hours of treatment with the IL-13 conjugated liposomes containing DXR. Cytotoxicity is calculated as a percentage absorbance at 490 nm after treating the cells with MTS/PMS dye. Notably, media from the apical chamber is several fold more cytotoxic to the glioma cells, indicating that the transport of DXR encapsulated liposomes does not occur by compromising the endothelial cells. Thus, the experiment provides evidence for effective transport of intact liposomes.

### Example 9

### Liposomal Binding and Internalization

Liposomes are conjugated to IL-13 and its mutants IL-13.E13K and IL-13.K105R as described. Further, liposomes are prepared with rhodamine phosphatidylethanolamine to observe internalization of the liposomes (Torchilin, V. P. et al. (2001) PNAS USA 98, 8786-8791). Conventional and confocal fluorescence microscopy are used to visualize binding and the internalization pattern of rhodamine fluorescence at various intervals of time. Thus, surface binding and receptor-mediated endocytosis of liposomes is monitored. For quantitative comparison of uptake of the liposomes by the IL-13Rα2 receptor, the liposomes (0.1 mM) are incubated with U251 glioma cells for two hours. The proportion of liposomes bound to the cell surface is calculated and the internalization of the liposomes is characterized by the first-order endocytosis rate constant (Equation A) (36).

Ke=(d[L]ᵢ/dt)ₛₛ/[L]_{s,ss} (A)

Where [L]ᵢ is the amount of internalized liposomes (per unit cell concentration), [L]s is the amount of cell surface bound liposomes and d[L]ᵢ/dt)ₛₛ is the liposome uptake rate at steady state.

### Example 10

### Cytotoxicity Assay

The chemotherapeutic drug, DXR, is encapsulated in liposomes conjugated with IL-13 and/or its high affinity mutants by a remote-loading method using ammonium sulfate (Abra, R. M. et al. (2002) Journal of Liposome Research 12, 1-3; Stevens, P. J. et al (2003) Anticancer Research 23, 439-442). DXR is encapsulated in liposomes conjugated with a variable molar proportion of IL-13 and/or high affinity mutants thereof to determine the most suitable combination to achieve higher specificity towards glioma cells. The U251, U87 and HUVEC cells as controls are plated in 96-well cell culture plates at 5×10³ cells/well and serially diluted conjugated liposome formulations with and without encapsulated DXR are added to the cells. Forty-eight hours after the addition of liposome formulations, cell viability is assessed with the MTS/PMS colorimetric assay (Cory, A. H., et al. (1991) Cancer Communications 3, 207-212), which assesses mitochondrial activity in the cells. Another set of cytotoxicity experiments is repeated with liposomes unconjugated to any IL-13 targeting moiety, and containing DXR, as a negative control.

### Example 11

### Assessment of Ability of IL-13 High-Affinity Mutant Conjugated Liposomes of Appropriate Size to Cross the Blood-Brain Barrier Efficiently

Assessment of ability of liposomes of a size range of 50 to 150 nanometers conjugated with IL-13 or its mutants, IL-13.K105R and IL-13.E13K, to be transported across the EBB is performed using a model of the blood-brain barrier (BBB) as described. The rate of transport of liposomes conjugated with wild type IL-13 is compared to the rate of transport of liposomes conjugated with IL-13 mutants. Dextran labeled with the fluorescent dye RITC is loaded simultaneously as a negative control to ensure that the treatments do not compromise the in vitro BBS. Once the rate of transport is established, liposomes encapsulating the cytotoxin DXR are used to show that the liposomes transport DXR across the EBB. Cytotoxicity assays are used to show that the transported DXR remains toxic to glioma cells and that the presence of high-affinity mutant IL-13 on the liposomes does not diminish the cytotoxicity or binding to the glioma cells.

### Example 12

### Blood-Brain Barrier Model

A EBB cell culture model is used as described herein. In one configuration, a BBS model is arranged in tissue culture wells (12 mm diameter, 0.4 μm pore size with a tissue culture treated polyester membrane) that utilize bovine retinal endothelial cells (BREC) as a layer of endothelial cells that is a replica of the BBB. Wild type and mutant IL-13 conjugated liposomes may be conjugated to FITC in order to quantify the transport. These liposomes are placed in the apical chamber of the EBB model system. Every two hours an aliquot of the media is removed from the basal chamber for a total of ten hours. The kinetics of transport is determined by measuring the fluorescence of the transported FITC conjugated liposomes at the excitation wavelength of 490 nanometers and emission at 555 nanometers using a fluorescent plate reader and the rate of flux (P₀) is calculated using the formula (Chang, Y. S., et al. (2000) Microvascular Research 59, 265-277):

P₀=[(F_{A}/Δt)V_{A}]/F_{L}A

Where P₀ is diffusive flux (cm/sec), F_{A} is the basal fluorescence, F_{L} is the apical fluorescence, Δt is the change in time, A is the surface area of the filter in square cm and V_{A} is the volume of the basolateral chamber in cubed centimeters.

### Example 13

### Cytotoxicity Assay

Cytotoxicity experiments are performed with the media collected from the basal chamber of the BBB model system. First, 2.5×10³ U251 glioma cells per well are plated in a 96 well plate in a total volume of 150 microliters. After a 24-hour incubation, 50 microliters of basal media or apical media from each collected time point is added to the cells and incubated for 48 hours. At the end of the incubation, the number of proliferating cells is determined by the colorimetric MTS/PMS assay (Promega. Madison, Wis.) that measures the absorbance at 490 nanometers. Cells treated with BSA and cycloheximide serve as positive and negative controls for the cytotoxicity assay.

### Example 14

### Therapeutic Efficacy and Biodistribution of Targeted Liposomes in Tumor Bearing Animal Models

G26-IL-13Rα2 cells are implanted into syngeneic immunocompetent mice as a tumor-bearing animal model (Mintz. A. et al. (2003) J. of Neuro-Oncology 64, 117-123). This cell line is the G26 mouse cell line transfected with IL-13Rα2 and it readily forms tumors in these mice. IL-13 and its mutants bind effectively to these G26-IL-13Rα2 cells. Mice bearing implanted tumors are administered DXR alone or liposome encapsulated DXR as well as drug free carrier solution or blank liposomes as controls. Single-dose response treatments include 0, 20, 35, and 50 mg/kg free and liposomal DXR (n=4 animals for each treatment). Multiple-dosing schedules include (a) 40 mg/kg free and liposomal DXR on day 14 after tumor inoculation followed by 20 mg/kg free and liposomal DXR 7 and 14 days later and (b) 20 mg/kg free and liposomal DXR 14 days after tumor inoculation followed by 40 mg/kg free and liposomal DXR 7 and 14 days later (n=8 animals for each treatment). Animals are divided into four groups for toxicological studies as follows: (a) untreated animals which serve as controls, (b) animals treated with IL-13 conjugated liposomes without drug, (c) animals treated with standard DXR formulations in saline, (d) animals treated with high affinity IL-13.K105R liposomes loaded with DXR, and (e) animals treated with tumor specific IL-13.E13K liposomes loaded with DXR.

### Example 15

### Tumor and Blood Analysis

Seven days after the mice are implanted with G26-IL-13Rα2 cells, when the tumors are palpable, the mice are injected intravenously with free DXR, IL-13 conjugated liposomal DXR. IL-13 mutant conjugated liposomal DXR and/or unconjugated liposomal DXR (6 mg/kg). Tumor growth is monitored by measuring perpendicular diameters (a and b), and the volume is calculated with the formula v=0.4 ab², where b>a. Treatment is also performed using unconjugated and IL-13 conjugated liposomes carrying DXR that have the same mean diameters (polydispersity). Before and during the course of chemotherapy, blood is collected from the tail veins of the mice. White blood cells and platelets are counted, and complete blood cell analysis is performed by differential microscopic analysis. In addition, animal weight is monitored daily throughout each treatment and necropsy examination of animals is performed.

### Example 16

### Pharmacokinetics and Biodistribution

The tumor bearing mice are anesthetized and a femoral vein is cannulated and injected with 0.001M PBS containing 4 micro Curies of free [³H] DXR or [³H] DXR loaded IL-13-liposomes. Blood samples are collected at various time intervals (0.25 to 60 mm) after injection of the isotopically labeled and liposome encapsulated DXR. After 60 minutes, the animals are killed to remove the heart, lung, liver, spleen, kidney, brain and tumor. In some case animals are killed 6 or 24 hours after injection. In this case, animals are allowed to recover from surgery and only terminal blood is sampled. The plasma and organ samples are weighed, solubilized and neutralized before liquid scintillation counting. Pharmacokinetic parameters are calculated by fitting plasma radioactivity data to a biexponential equation

A(t)=A₁e^{-k}1^{t}+A₂ e^{-k}2^{t}

where A(t)=% ID/ml of plasma [³H]radioactivity (% ID, percent injected dose).

The biexponential equation is fit to plasma data using a non-linear regression analysis. This quantitative determination of the efficacy with which IL-13 conjugated liposomes bind to the IL-13Rα2 in glioma tumors as well as evaluate any systemic toxicity.

### Example 17

### Conjugation of Targeting Moiety to Liposomes

The targeting moiety protein is modified according to the method reported by Shaik et al (Shaik, M S, Kanikkannan, N., Singh, M. J. Controlled Release, 2001, 76, 285-295). SPDP (Succinimidyl-6-[3-(2-pyridyldithio)propionamido)hexanoate) is used to introduce the pyridyl disulfide groups into the IL-3 or IL-13 mutant molecule. 10 mol of SPDP is reacted with 1 mol of IL-13 protein in phosphate buffered saline for 24 hours. Then the unreacted SPDP is removed by dialyzing against PBS using a dialysis bag of molecular weight cut off 10000. Then subsequently they are reduced with DTT (dithiothreitol) and unreacted DTT is removed by passing the mixture through a column of sephadex G-25M column. The thiolated IL-133 and N-[2-Pyridyldythio)propionyl]-stearylamine (PDP-SA) are reacted for 14 h at 4° C. Then the modified liposomes are separated and purified by ultracentrifugation (50000 rpm) for 45 mm and washing with PBS.

### Example 18

### Method of Encapsulating a Therapeutic and/or Diagnostic Agent in Liposomes

Doxorubicin is encapsulated into the liposomes by ammonium sulfate gradient method (Hansen, C. B. et al. (1995) BBA, 1239, 133-144). The liposomes are hydrated with ammonium sulfate pH 5.5 (155 mM) using a sonicator. The concentration of phospholipid is maintained at 10 mM. The external buffer is exchanged by passing the liposomes through Sephadex G-25M column and eluting them with 123 mM sodium citrate, pH 5.5. Then the liposomes are incubated with doxorubicin (0.2 mg DXR per mg phospholipid) for 1 h at 65° C. Unencapsulated doxorubicin is removed by passing the liposomes through Sephadex G25M column and exchanging them with PBS.

### Example 19

### Preparation of Liposomes

1,2-dipalmitoyl-sn-glycero-S-phosphocholine (DPPC), cholesterol, 1,2-distearoyl-sn-glycero-3-phosphoethanolamine-N[carboxy(polyethylene glycol 2000] are purchased from Avanti Polar Lipids, Alabaster, Ala. Lipids and stearylamine are purchased from Aldrich.

Liposomes are prepared from DPPC, cholesterol, DSPE-PEG 2000 and stearylamine in a ratio of 10:5:2.5:2.5 by lipid film hydration.

The resulting multilamellar liposomes are extruded 10 times at room temperature through two stacked 0.1 micron polycarbonate membranes. The size of the liposomes is measured by a dynamic laser light scattering method. For microscopy studies, 0.1 mol % of fluorescently labeled phospholipids (Rho-PB) is added to the lipid mixture. The liposomes are stored in HEPES-buffered saline at 4° C.

### Example 20

### Preparation of IL-13 Conjugated Liposomes

N-[3-(2-pyridylthio)propionyl]-stearylamine (PDP-SA) is prepared by the method of Singh et al. Liposomes are prepared using DPPC:CHOL:DSPE-PEG:SA:PDP-SA in the molar ratio of 10:5:2.5:2.5:1.5 in a manner similar to that described infra. The heterobifunctional agent SPDP is employed to introduce pyridyldisulphide groups into the IL-13 molecule by reacting SPDP with IL-13 in the molar ratio of 10:1 for 24 hours. This is then further treated with dithiothreitol and purified by eluting the mixture through a Sephadex G25 M column.

The resulting modified IL-13 is treated with liposomes for 24 hours at 4° C. The liposomes are then purified by centrifuging at 50,000 rpm and subsequent washing with PBS. The immunoreactivity of IL-13 after conjugation to liposomes is verified by dot blot on a nitrocellulose membrane.

### Example 21

### Method of Conjugation of a Targeting Moiety to a Liposome

Lipids like MPB-PE or MCC-PE may be included in the liposome composition for conjugation to a targeting moiety. Thus, an MPB lipid, such as 18:1 MPB-PE (1,2-Dioleoyl-sn-Glycero-3-Phosphoethanolamine-N-[4-(p-maleimidophenyl)butyramide)and 16:0 MPB-PE (1,2-Dipalmitoyl-sn-Glycero-3-Phosphoethanolamine-N44(p-maleimidophenylbutyramide) or an MCC lipid, illustratively including 18:1 MCC-PE(1,2-Dioleoyl-sn-Glycero-3-phosphoethanolamine-N[4(p-maleimidomethyl)cyclohexane-carboxamide) and 16:0 MCC-PE (1,2-Dipalmitoyl-sn-glycero-3-phosphoethanolamine-N-[4-(maleimidomethy)cyclohexane-carboxamide) may be included in a lipid composition for forming liposomes useful in compositions and methods according to the invention. These and other lipids are commercially available from suppliers such as Avanti Polar Lipids, Alabaster, Ala. In such a method, a thio containing protein and/or peptide can be conjugated to the liposomes. MPB and MCC lipids have advantages of being stable complexes that can survive in serum longer (see, for instance, Martin, F. J., and Papahadjopoulos, D. (1982) J. Biol. Chem. 257, 286-288) and MCC contains the more stable maleimide function group towards hydrolysis in aqueous reaction environments (see, for instance, Hashida S., and Ishikowa, B. (1985) Anal. Lett. 18(b9), 1143-1155; Dewey, R. E., Timothy, D. H., and Levings III, C. S. (1987) A mitochondrial protein associated with cytoplasmic male sterility in the T cytoplasm of maize. Proc. Natl. Acad. Sci. U.S.A. 84, 5374-5378).

In one example, liposomes are prepared according to the general procedures described herein and in cited references from DPPC, cholesterol, DSPE-PEO 2000 and stearylamine and MCC (1,2-Dipalmitoyl-sn-glycero-3-phosphoethanolamine-N-[4-(p-maleimidomethyl)cyclohexane-carboxamide]) in a ratio of 10:5:2.5:2.5:0.5.

1-2 micromoles of phospholipids in this ratio are mixed with thiolated reduced IL-13 protein (0.5-2 mg/ml final concentration). The resulting liposomes include MBP-PE bound to IL-13 as shown in the reaction scheme below. IL-13 mutants may also be bound to MBP-PE.

### Example 22

### In Vivo Delivery of Cargo Moiety and Treatment of Tumors

The therapeutic efficacy of the IL-13 receptor targeted liposomes carrying doxorubicin was tested in a subcutaneous glioma tumor model in nude mice. (See, FIG. 9). Mice were given intraperitoneal injections once a week. The insert shows that mice receiving targeted liposomes with doxorubicin had a greater reduction in tumor size in the first two weeks compared to the animals receiving the same concentration of unconjugated liposomes and doxorubicin. The tumors of the other groups increased during the initial three weeks of the injections. The main figure shows the pattern of the tumor growth over 7 weeks of injections of liposomes (LIP) containing doxorubicin (DXR) at the indicated concentrations or liposomes without drug (LIP without DXR). The tumor volume is plotted as a mean and standard error. The error bars on the LIP (DXR) 15 mg/kg group are contained within the symbol for this group.

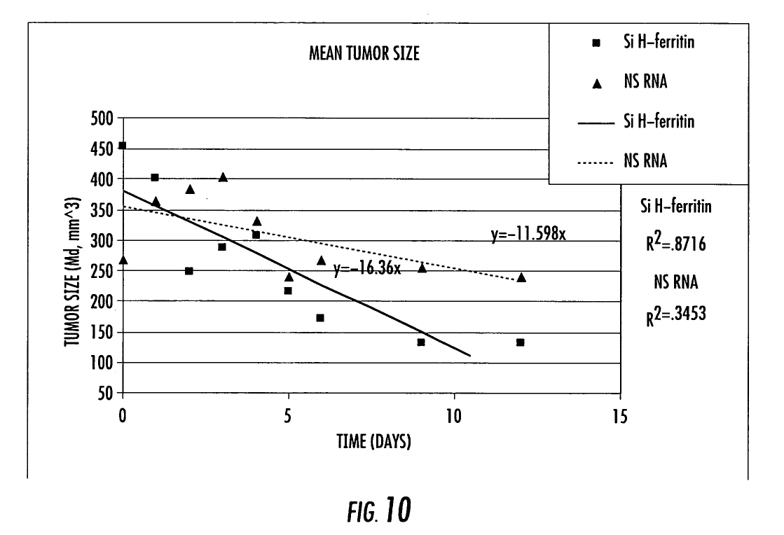

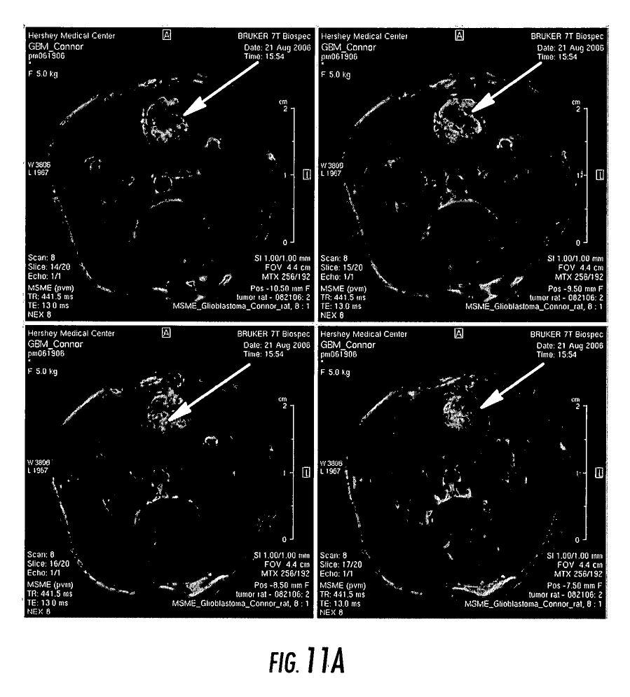

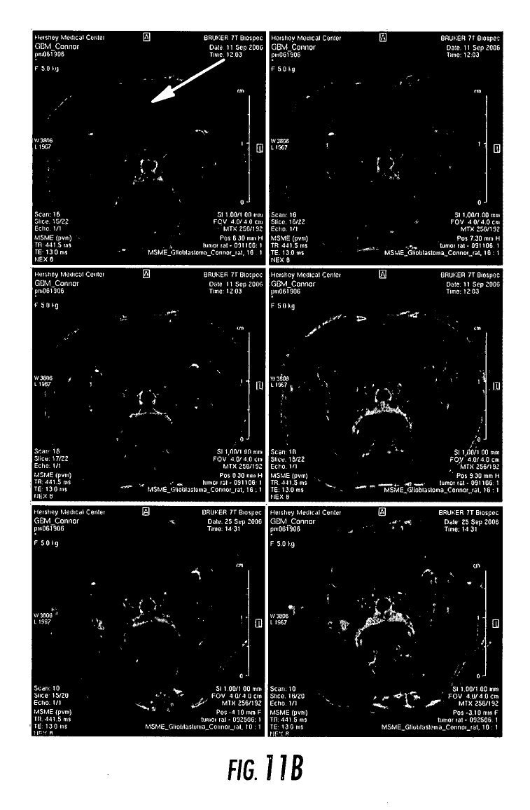

The results obtained in vivo with siRNA H-Ferritin are shown in FIG. 10. For this study, a subcutaneous tumor model was used to show the in vivo efficacy of the siRNA H-ferritin approach. The siRNA for H-ferritin or the nonsense (NS) control was first conjugated into liposomes and then injected directly into a subcutaneous glioblastoma tumor growing in the flank of nude mice. The concentration of siRNA or NS RNA injected into the tumor was ˜4 μg. After injection of the siRNA, the mice, received 25 μM of BCNU delivered i.p. 24 hours. The injections were performed once a week. As can be seen in this figure, the rate of tumor shrinkage was significantly faster in the animals receiving siRNA in the tumors as opposed to NS RNA. The significance of the data in this graph are two-fold: 1) the data provide proof of concept that siRNA for H-ferritin delivered into tumors will enhance the efficacy of standard chemotherapeutic agents, 2) the siRNA can be delivered to the tumors using a liposome delivery system.

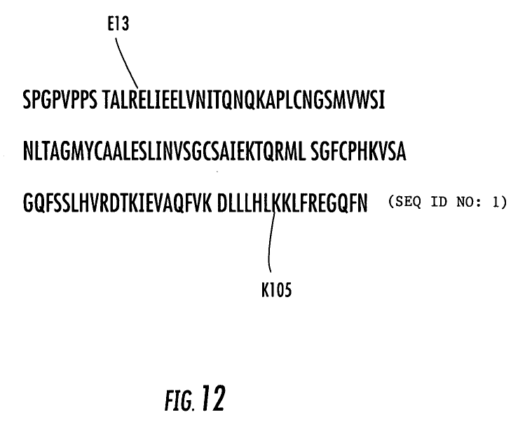

Intravenous delivery of targeted nanovesicles as an effective model to treat intracranial tumors is shown in FIGS. 11A and 11B. FIG. 11A shows images of a tumor (bright spot indicated by the arrow) in a rat 3 weeks after surgery to implant the tumor cells. The animal has not received any treatments. FIG. 11B shows treatment with Il-13 conjugated liposomes delivering doxorubicin. The liposomes were injected intravenously. The top 4 panels are images from the same rat in FIG. 11A after 2 injections over 3 weeks of IL-13 conjugated liposomes delivering doxorubicin (15 mg/kg). The bottom 2 images are also from the same rat after a third injection and 5 weeks post treatment. The arrow indicates the location of where the tumor had been.

### Example 23

### Interleukin-13 Receptor Targeted Nanovesicles are a Potential Therapy for Glioblastoma Multiforme

The anti-tumor effect of doxorubicin encapsulated IL13 conjugated liposomes was evaluated on a subcutaneous tumor mouse model.

Abbreviations-DXR:doxorubicin, IL13:Interleukin-13, Pgp:P-glycoprotein, SPDP:(N-succinimidyl-3(2-pyridyldithio)propionate), DSPE-PEG: 1,2-Distearoyl-sn-Glycero-3-Phosphoethanolamine-N-[Carboxy(Polyethylene Glycol)2000 (ammonium salt), DPPC: Dipalmitoylphosphatidylcholine, Rho-PE: L-α-Phosphatidylethanolamine-N-(lissamine rhodamine B sulfonyl)(ammonium salt), GBM: Glioblastoma Multiforme, HGA: High-grade Astrocytomas, EEA1:Early Endosomal Antigen.

### Materials and Methods

1,2-Distearoyl-sn-Glycero-3-Phosphoethanolamine-N-[Carboxy(Polyethylene-Glycol)2000 (ammonium salt)(DSPE-PEG), Dipalmitoylphosphatidylcholine (DPPC), Cholesterol (CHOL), and L-α-Phosphatidylethanolamine-N-(lissamine rhodamine B sulfonyl)(ammonium salt) (Rho-PE) were purchased from Avanti Polar Lipids (Alabaster, Ala., USA). Stearylamine was purchased from Sigma Chemical (St. Louis, Mo., USA). Human U251 and U87 glioma cells were purchased from American Type Tissue Culture collection. Doxorubicin was obtained from Sigma Chemicals (St. Louis, Mo., USA). Coming 96-well plates were purchased from Corning (Corning, N.Y., USA). Cyclosporine A was purchased from Calbiochem (La Jolla, Calif.). Early endosomal antigen antibody EEA1 was from Santa Cruz Biotechnology, Santa Cruz, Calif.

Preparation and characterization of IL13 conjugated liposomes: Sterically stable liposomes were formulated using DSPE-PEG, DPPC, Cholesterol, and modified Stearylamine (PDP-SA) were dissolved in methanol:chloroform mixture (2:1) in a molar ratio of DPPC: CHOL:DSPE-PEG:PDP-SA=10:5:1.5:1.5. The liposomes were subsequently rotoevaporated to obtain a lipid film and the lipid film was further dried in a dessiccator. For doxorubicin encapsulation or HEPES buffer for binding studies, the lipid film was hydrated in 155 mM ammonium sulfate pH 5.5 and then sonicated in a bath type sonicator for 15 minutes. In order to fluorescently tag the liposomes for the cellular uptake and the liposomes were constructed using 1 mol % of fluorescently labeled phospholipid (Rho-PE). A polycarbonate membrane of gradually decreasing pore size was used to produce small unilamellar vesicles (SUV) by extruding through two-stacked 0.1 μm polycarbonate membrane and subsequently with 0.05 μm polycarbonate membrane using a nitrogen pressure operated extruder (Lipex extruder, Northern lipids Inc., Canada). All the extrusions were performed at an operating pressure of 800 psi (5440 kPa). The liposomes were then purified and sterilized by passing through Sephadex G25M column. The liposome concentration was determined by phosphate assay. The size distribution of the liposomes was determined by dynamic light scattering which was conducted using an ALV/DLS/SLS-5022F compact goniometer system (ALV, Germany), which was confirmed by Transmission Electron Microscope (TEM) using uranyl acetate as the staining agent.

Human IL13 gene, which was isolated from the total RNA (BD Biosciences, Mountain View, Calif.) from human testis by RT-PCR, which was cloned into the TOPO-vector (Invitrogen, Carlsbad, Calif.), expressed in E. coli as His-tagged protein and purified by nickel affinity binding. Conjugation of IL13 to liposomes was performed according to the method reported by Singh et al (European Journal of Pharmaceutics & Biopharmaceutics 2001;52(1):13-20). An heterobifunctional reagent SPDP (N-succinimidyl-3(2-pyridyldithio)propionate) was employed to introduce pyridyl disulphide groups to the IL13 molecule. Briefly, 10 mol of SPDP was dissolved in methanol and then this solution was reacted with 1 mol of IL13 in PBS for 24 hours at 4° C. The unreacted SPDP was removed by dialysis against PBS (MWCO 10000). The dialysate was reduced with DTT (20 mM final concentration) and the unreacted excess DTT was removed by gel filtration through a Sephadex G25M column. Thiolation of IL13 was verified by the presence of free sulfhydryl groups which were estimated by Ellmann's method according to Ellman's reagent protocol (Pierce, USA).

Thiolated IL13 protein was slowly added to a 5 mL beaker containing the liposomes and a magnetic stirring bar and incubated overnight with slow stirring at 4° C. The conjugated liposomes were separated by ultracentrifugation at 40000 rpm. The IL13 to phospholipids mole ratio was maintained at 1:700. After conjugation the presence of IL13 protein on the liposomes were verified by Coomasie (Bradford) protein binding assay. The lipid content of the liposome was measured by phosphorus estimation according to the method of phosphorus determination by Morrison (Analytical Biochemistry 1964;7:281-4).

To add transferrin to the liposomes, commercially available bovine Tf (Sigma) was conjugated to IL13 liposomes by a similar method to that described for IL13. SPDP modified Tf protein was reduced with DTT for thiolation. Thiolated Tf was reacted with IL13 conjugated liposomes using a phospholipids to Tf mole ratio of 1:700 using the same methods and conditions as that for IL13 conjugation.

Method of encapsulation of doxorubicin in the liposomes: Doxorubicin was encapsulated into the liposomes by ammonium sulfate gradient method (Hansen C B, et al. Biochim Biophys Acta 1995;1239(2):133-44). The liposomes were hydrated with ammonium sulfate pH 5.5 (155 mM) using a bath type sonicator. The liposomes were then extruded as before. The concentration of phospholipid was maintained at 10 mM. The external buffer was exchanged by passing the liposomes through Sephadex G-25M column and eluting them with 123 mM sodium citrate, pH 5.5. Then the liposomes were incubated with doxorubicin (0.2 mg DXR per mg phospholipid) for 1 h at 65° C. In all our preparations, the drug to lipid weight ratio was maintained to be 1:5. Unencapsulated doxorubicin was removed by passing the liposomes through Sephadex G25M column and exchanging them with PBS.

Doxorubicin leakage study: The leakage of DXR from the IL13 conjugated liposomes and the unconjugated liposomes was determined by suspending 10 μl of DXR containing liposomes in 0.5 ml of human serum or dialyzed against a large volume of PBS. Both experiments were performed at 37° C. for increasing time intervals. Both serum and PBS media were evaluated to compare shelf life (PBS) and in vivo stability for delivery (serum). To determine the amounts of DXR that may have been released from the liposomes, the serum samples were centrifuged at 40000 rpm and the supernatants were analyzed for doxorubicin by measuring the absorbance at 492 nm. For the PBS studies, the dialysate was collected and assayed for DXR.

Uptake of IL13 conjugated liposomes in normal and glioma cells: Uptake of the IL13 conjugated liposomes on glioma cells was performed to investigate the ability of the glioma cells to internalize the liposomes. Both U251 and U87 glioma cells (10,000 cells each) were cultivated on a chamber slide for 24 h. IL13 conjugated rhodamine labeled liposomes were added for 120 min at 37° C. Human umbilical vein endothelial cells (HUVEC) and SVG p12 glial cells (purchased from ATCC) served as controls. The SVG p12 cell lines are human fetal glial cells from brain material, which are transfected with DNA from an ori-mutant of SV40. The cells were washed 3 times with PBS to end the exposure to liposomes and then viewed with confocal microscope. The cells were stained with DAPI to visualize the nuclei.

To determine if the uptake of the liposomes involved the endosomal system, U251 glioma cells were cultured on chamber slides as described above and the cells were permeabilized and blocked for 30 min in 0.1% BSA and PBS (blocking buffer). The cells were treated with rhodamine labeled IL13 conjugated liposomes and then stained with polyclonal EEA1 antibody (1:15) for 30 min. The cells were then washed 3 times with PBS and counterstained with FITC-antigoat antibody (1:75) for 30 min and observed under fluorescent microscope. The images were captured using a digital camera.

Flow cytometry: Flow cytometry was used to measure total intracellular doxorubicin fluorescence. In this report we refer to fluorescence intensity as intracellular drug content. 1×10⁶ cells were exposed to 20 μM of drug as (a) free DXR (b) DXR encapsulated IL13 conjugated liposomes (c) DXR encapsulated unconjugated liposomes for 2 h. All drug treatments and post treatment incubations were performed in complete growth medium. The cells were washed to remove any free adherent DXR using PBS and centrifuged. Cells were released from tissue culture dishes with 0.05% trypsin/0.02% EDTA followed by PBS washing (centrifugation, 5 min 500 g) and resuspended in PBS for flow cytometry assay. The intracellular accumulation of inherently fluorescent doxorubicin was evaluated using a fluorescence activated cell analyzer. A single 15 mW argon ion laser beam (488 nm) was used to excite the fluorescence of DXR. A total of 10000 cells were analyzed for each histogram. Experiments were repeated 3 times and the fluorescence intensities of DXR were expressed in arbitrary units.

Binding to human brain tumor sections: To demonstrate the potential clinical application of the conjugated liposomes, we obtained Glioblastoma Multiforme and Pilocytic Astrocytoma brain tumor sections and exposed them to the rhodamine labeled IL13 liposomes. Brain tumor samples were obtained from patients undergoing surgical decompression at Penn State University Hershey Medical Center. All studies involving human specimens were approved by the respective Human Subjects Protection Office at the Penn State College of Medicine (Protocol. No. 96-123EP). Serial tissue sections were generated (10 μm) on a cryostat, thaw mounted on chromalum coated slides, and stored at −70° C. until analyzed. The sections were then blocked with normal goat serum (10%) and then exposed to rhodamine labeled IL13 conjugated liposomes for 1 h at 37° C. and they were washed 3 times with PBS before observing them via fluorescence microscopy. In order to test the hypothesis that the IL13 conjugated liposomes interacted with the IL13 receptor on the GBM tumors, some of the GBM sections were blocked with 1 mg/ml concentration of IL13Rα2 receptor antibody and followed by exposure to rhodamine labeled IL13 conjugated liposomes. The sections were then washed with PBS and observed under fluorescence microscope.

Effect of Pgp inhibitor on the internalization of IL13 conjugated liposomes in the glioma cells: About 50000 U251 glioma cells were plated in a small petridish and were exposed to either IL13 conjugated liposomes carrying 20 μM of doxorubicin or the same concentration of free doxorubicin (20 μM) for 2 hours. The cells in each condition were either treated or not with cyclosporine A, a Pgp inhibitor (5 μg/ml) for 30 mins prior to addition of the liposomes. After 2 h of incubation, they were washed with PBS and the cells were removed with versene and subjected to flow cytometry.

Cytotoxicity assay with DXR encapsulated ligand-targeted liposomes: In our experiments we used DXR encapsulated liposomes, which are unconjugated, conjugated with IL13 or double conjugated with IL13 and Tf to determine their cytotoxic potential. The cytotoxicity was measured after adding serially diluted DXR encapsulated liposomes to U251 glioma cells plated in 96-well cell culture plates at a concentration of 5×10³ cells/well. Cell survival was determined after 48 h by MTS/PMS assay. Cells treated with high concentrations of cycloheximide served as the background for the assay.

In vivo therapeutic efficacy of targeted liposomes: To test the in vivo efficacy of the targeted liposomal system, adult female athymic nude mice were implanted in the flank subcutaneously with U251 glioma cells. Exponentially growing cells were harvested and 15×10⁶ cells per mouse were subcutaneously injected. After 2 weeks, a tumor of volume 14-30 mm3 was observed. At that time the mice were divided into 5 different groups of 6 mice in each group. One group of mice was injected with IL13 conjugated liposomes carrying doxorubicin at a dosage of 15 mg/kg body weight. A second group was injected with the same amount of liposomes carrying 15 mg/kg body weight of doxorubicin, but these liposomes were unconjugated. A third group was received injections of IL13 conjugated liposomes but with a lower dosage of doxorubicin (7.5 mg/kg body weight). The fourth group of mice were untreated and received injections of 0.1M phosphate buffered saline as a control. A fifth group of mice were injected with unconjugtaed liposomes carrying no drug as an additional control. All the drugs were administered intraperitoneally once a week. The injections were given opposite the side of the subcutaneous tumor. The tumor size, health and survival of the mice were monitored once a week by an investigator (BW) blinded as to the groups of mice. These experiments were approved by the Pennsylvania State University IACUC.

### Results

Liposome composition and particle size: The particle size of the liposome as confirmed by laser particle size analyzer and TEM was found to be in the range of 50-150 nm with a mean size of 104 nm. The polydispersity index for various batches of nanovesicles consistently lies in the range of 0.2-0.4. After conjugation and purification the concentration of the phospholipids in the liposome was 21.8 μg of phospholipids/μl and the concentration of IL13 conjugated on the liposomes after doxorubicin encapsulation was 3.46×10⁻⁷ μmol of IL13/μg of phospholipids. The final concentration of DXR is 0.18 μg per μg of phospholipid.

We also observed the effect of temperature on encapsulation efficiency of the drug DXR to be maximum (90%) at 65° C. when compared to lower temperatures 25° C. and 40° C. where the encapsulation efficiencies are 45% and 72% respectively. The T_{1/2} for DXR leakage from IL13 liposome at 37° C. in PBS was 25 days whereas with unconjugated liposomes it is approximately 45 days. Thus the IL13-conjugated liposomes were not substantially leaky during the experimental period, because our experiments were performed within 2 weeks of preparation. We did not observe any significant leakage of DXR from the liposomes that were incubated in human serum at 37° C. for at least one week.

Binding to glioma cells: In order for IL13 receptor targeted liposomes to be considered for clinical use, it is necessary to show that glioma cells take up the liposomes. Uptake of the liposomes is seen in both U87 and U251 glioma cell lines, whereas normal cells like HUVEC and the immortalized glial cell line SVGp12 which do not overexpress IL13 receptor had no detectable uptake over the same exposure time.

Intracellular accumulation and retention of DXR in U251 glioma cells: The uptake and accumulation of the IL13 conjugated liposomes was analyzed using flow cytometry and fluorescent microscopy. The IL13 conjugated liposomes enter early endosomes as demonstrated by co-localization with early endosomal antigen (EEA1). The relative accumulation of the DXR in U251 cells depending on the mode of delivery was demonstrated by flow cytometery. The ability to demonstrate DXR in cells by FACs analysis takes advantage of the intrinsic fluorescence of DXR when excited at 488 nm. The flow cytometry analysis showed a right shift in the curve indicating an increase in the cell fluorescence in U251 glioma cells after exposure to free DXR or liposomal DXR. The right shift is greater with IL13 conjugated liposomal DXR than with non-conjugated liposomes. Drug accumulation in the cancer cells is decreased by Pgp activity. When doxorubicin is delivered by IL13 conjugated liposomes the intrinsic fluorescence of the doxorubicin accumulated or retained intracellularly in U251 glioma cells is much higher than that seen in cells exposed to free doxorubicin. Indeed, the level of DXR detected in the cells following delivery via liposome was even greater than that seen when the cells treated with free DXR which were also exposed to cyclosporine A, a P-glycoprotein inhibitor.

Exposure of glioma tumors to liposomes: Representative samples of GBM and Pilocytic Astrocytomas and normal human cortex exposed to IL13-conjugated liposomes tagged with rhodamine show a much greater affinity of the GBM and pilocytic astrocytoma samples for the IL13 conjugated liposomes than the medulloblastoma or normal human cortex samples. The specificity of this association of IL13 conjugated liposomes to the IL13 receptor was demonstrated by exposing the tumor sections to IL13 receptor antibody followed by IL13 conjugated vesicles. This approach resulted in a decrease in the binding of IL13 conjugated rhodamine labeled liposomes.

Cytotoxicity assay with ligand targeted liposomes: The cytotoxicity of DXR on U251 glioma cells encapsulated in IL13 conjugated liposomes versus unconjugated liposomes was compared and the results shown in FIG. 14. Because we were also evaluating the possibility of using liposomes doubly conjugated with Tf and IL13, these liposomes were also included in this experiment. The concentration of liposomes, which were added to each of the cell cultures, was identical and each liposome carried equal amounts of doxorubicin. At the lowest concentration, 150 ng/ml of liposomal DXR, IL13 conjugated liposomes were 31.7% more cytotoxic than unconjugated liposomes (p<0.001). The cytotoxicity of the doubly conjugated (IL13, Tf) liposomes was similar to the IL13 conjugated liposomes (35.3%). With increasing concentration, the liposomal doxorubicin cytotoxicity increases and increases at a faster rate than the cytotoxicity associated with the unconjugated liposomes.

In vivo anti-cancer therapeutic efficacy: The tumors in the control mice grew from 14 mm³ to 570 mm³ in seven weeks, whereas the tumor growth rate is much lower in those animals that received doxorubicin carrying liposomes. The most effective approach at reducing the tumors was IL13 conjugated liposomes carrying doxorubicin (15 mg/kg body weight). In this group the tumor volume decreased by 69% over the first two weeks following injections. The only other group to show an initial decrease in tumor size (52%) was the one receiving injections of unconjugated liposomes carrying DXR. The group receiving the highest dose of the conjugated liposomes and DXR had a tumor volume of only 37 mm³ or less than 10% of the untreated group after 7 weeks (termination of the experiment). Animals receiving the same dose of unconjugated liposomes had a tumor volume of 192 mm³ in 7 weeks; 5-fold more than the animals receiving the same concentration of DXR in targeted liposomes and 22% higher than animals receiving the lowest dose of DXR in conjugated liposomes (see, FIG. 9). In the group that received only liposomes (untargeted and not containing DXR) the tumor volume did not decrease appreciably and at the end of 7 weeks had an average volume of 452 mm³. During the course of these studies, only 1 animal died. This animal was in the high DXR group (conjugated liposomes) and the death appeared related to an injection artifact. No animals died in the other groups.

Discussion: Previously, IL13 receptors have been identified as a potential target on HGA, but the outcomes have been mixed. Here we show an alternative approach of using IL13 conjugated liposomes to selectively target and deliver the cytotoxin DXR to tumor cells that is effective in both in vivo and cell culture models. The liposomes in this study have a mean size of 104 nm. This size is optimal for nanoparticles to cross the BBB and also smaller liposomes have a relatively extended half life. In addition, the antitumor activity of the liposomal doxorubicin is sensitive to vesicle size and the liposomes in this size range can readily release their contents within the cells; which is consistent with our observations in this study.

Our liposome system is composed of PEG lipids which provide a steric barrier at the liposome surface, inhibiting protein binding and therefore opsonisation. The ligand IL13 is conjugated to the lipid portion rather than on the surface of the PEG moiety. Our data indicate that the liposomes constructed in this manner maintain their targeting property, maintain an ability to effectively encapsulate and retain the drug following covalent attachment of IL13, and are still able to bind the target IL13Rα2 on the glioma cells. Moreover, our liposomes are relatively, stable and unlike egg-phosphatidylcholine/cholesterol liposomes, after drug encapsulation, they are not leaky in serum or buffer at physiological temperatures. The liposomes configured in our study only became leaky at a temperature well above the transition temperature of DPPC.

Our study demonstrated effective binding of IL13-conjugated liposomes to the malignant cells and the clinical specimens of brain tumors in situ. We provided evidence for an affinity to high-grade astrocytoma (GBM) as well as a low-grade pilocytic astrocytoma. These observations are consistent with the presence of IL13Rα2 on these tumors. The uptake studies demonstrated that the liposomes were found in early endosomes, which is consistent with receptor mediated uptake. The lack of affinity of the liposomes for the normal human cortex or the HUVEC is consistent with the absence of detectable IL13Rα2 receptor.

The cytotoxicity experiments and in vivo experiments revealed that the IL13 conjugated liposomes were superior to the unconjugated liposomes at killing the tumor cells. Most brain tumors express P-gp which confers drug resistance to glioma cells. Our results showed that the liposome delivered DXR was not expelled by P-gp from the cell, unlike the unencapsulated DXR. Therefore, the explanation for the enhanced cytotoxicity with the IL13 targeted liposomes is that doxorubicin delivered by these liposomes results in increased accumulation and retention in glioma cells. The demonstration that liposome delivered DXR can both avoid expulsion from tumor cells in a cell culture model and have greater efficacy in the in vivo model strongly supports the notion that liposomal delivery is a viable option for brain tumors in vivo.

A critical component of drug delivery systems is their ability to target the tumors without adverse effect to the normal healthy tissues and to transport therapeutic agents into the tumors overcoming the Pgp mediated drug resistance. In our in vivo model we could clearly observe higher therapeutic efficacy of the IL13 conjugated liposomes where the tumor volume was reduced by 68% in 3 weeks, whereas in the unconjugated liposomes the tumor volume was only reduced by 50% over 3 weeks. The difference in final volume (7 weeks) between conjugated and non-conjugated liposomes (over 500%) is compelling evidence that IL13 conjugated liposomes carrying doxorubicin are much more efficacious than untargeted liposomes carrying same amount of doxorubicin. The cell culture data suggests that the greater efficacy of the targeted liposomes is a combination of the receptor targeting nature of the liposomes and the ability of the targeted liposomes to overcome the Pgp mediated drug efflux by the tumor. Thus, IL13 receptor targeted nanovesicles represents a viable approach where the liposomes of particle size range 50-150 nm can be utilized to deliver chemotherapeutic agents to brain tumor cells and may be a viable option for intravenous drug delivery applications across the blood-brain barrier.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art.

What is claimed:

1. A pharmaceutical delivery system comprising:
a particulate delivery vehicle having a wall, the wall defining an external surface and an internal volume; and,
a cargo moiety associated with the delivery vehicle.

2. The pharmaceutical delivery system of claim 1, wherein the delivery vehicle is a liposome.

3. The pharmaceutical delivery system of claim 2, wherein the liposome comprises: distearophosphoethanolamine polyethyleneglycol 2000 (DSPE-PEG), dipalmitoylphosphatidylcholine (DPPC), cholesterol (CHOL), and stearylamine (SA).

4. The pharmaceutical delivery system of claim 3, wherein the liposome further comprises MCC (1,2-Dipalmitoyl-sn-glycero-3-phosphoethanolamine-N-[4-(p-maleimidomethyl)cyclohexane-carboxamide]) and/or DSPE-PEG-Maleimide (1,2-Distearoyl-sn-Glycero-3-Phosphoethanolamine-N-[Maleimide(Polyethylene Glycol)2000 (Ammonium Salt)).

5. The pharmaceutical delivery system of claim 1, wherein the cargo moiety is at least partially localized in the internal volume of the delivery vehicle.

6. The pharmaceutical delivery system of claim 1, further comprising a targeting moiety conjugated to the external surface of the wall of the delivery vehicle.

7. The pharmaceutical delivery system of claim 6, wherein the targeting moiety is a ligand of a receptor present on a target cell.

8. The pharmaceutical delivery system of claim 6, wherein the receptor is preferentially expressed by a target cell compared to a non-target cell.

9. The pharmaceutical delivery system of claim 6, wherein the receptor is a human IL-13Rα2 receptor.

10. The pharmaceutical delivery system of claim 6, wherein the targeting moiety is human IL-13.

11. The pharmaceutical delivery system of claim 6, wherein the targeting moiety is a mutant of IL-13 which binds a human IL-13α2 receptor.

12. The pharmaceutical delivery system of claim 6, wherein the target cell is a tumor cell.

13. The pharmaceutical delivery system of claim 6, wherein the tumor cell is an astrocytoma cell.

14. The pharmaceutical delivery system of claim 6, wherein the mutant of IL-13 which binds a human IL-13Rα2 receptor binds to the IL-13Rα2 receptor with greater affinity than it binds to a wild-type human IL-13 receptor.

15. The pharmaceutical delivery system of claim 14, wherein the mutant of IL-13 is selected from the group consisting of: IL-13.K105R, IL-13.E13K and a combination thereof.

16. The pharmaceutical delivery system of claim 1, wherein the delivery vehicle has a diameter in the range of about 1-1000 nanometers.

17. The pharmaceutical delivery system of claim 1, wherein the delivery vehicle has a diameter in the range of about 50-150 nanometers.

18. The pharmaceutical delivery system of claim 1, wherein the cargo moiety comprises iron.

19. The pharmaceutical delivery system of claim 1, wherein the cargo moiety comprises an anti-cancer composition.

20. The pharmaceutical delivery system of claim 1, wherein the cargo moiety comprises an siRNA composition.

21. The pharmaceutical delivery system of claim 1, wherein the cargo moiety comprises an anti-ferritin siRNA composition.

22. A pharmaceutical composition comprising:
a plurality of particulate delivery vehicles, each particulate delivery vehicle having a wall defining an external surface and an internal volume, and each particulate delivery vehicle having a cargo moiety associated therewith and a targeting moiety conjugated thereto; and,
a pharmaceutically acceptable carrier.

23. The pharmaceutical composition of claim 22, wherein the plurality of particle delivery vehicles has a mean particle size in the range of about 1-1000 nanometers.

24. The pharmaceutical composition of claim 22, wherein the plurality of particle delivery vehicles has a mean particle size in the range of about 50-150 nanometers.

25. The pharmaceutical composition of claim 22 wherein the targeting moiety is selected from the group consisting of: human IL-13, an IL-13.K105R mutant of human IL-13, an IL-13.E13K mutant of human IL-13, and a combination thereof.

26. The pharmaceutical composition of claim 22, wherein the liposome comprises:
distearophosphoethanolamine polyethyleneglycol 2000 (DSPE-PEG), dipalmitoylphosphatidylcholine (DPPC), cholesterol (CHOL), and stearylamine (SA).

27. The pharmaceutical composition of claim 26, wherein the liposome further comprises MCC (1,2-Dipalmitoyl-sn-glycero-3-phosphoethanolamine-N-[4-(p-maleimidomethyl)cyclohexane-carboxamide]) and/or DSPE-PEG-Maleimide (1,2-Distearoyl-sn-Glycero-3-Phosphoethanolamine-N-[Maleimide(Polyethylene Glycol)2000 (Ammonium Salt)).

28. The pharmaceutical composition of claim 22, wherein the cargo moiety comprises iron.

29. The pharmaceutical composition of claim 22, wherein the cargo moiety comprises an anti-cancer composition.

30. The pharmaceutical composition of claim 22, wherein the cargo moiety comprises an siRNA composition.

31. The pharmaceutical composition of claim 22, wherein the cargo moiety comprises an anti-ferritin siRNA composition.

32. A liposome having human wild-type IL-13 or a mutant of human wild-type IL-13 having higher affinity for the human IL-13Rα2 receptor than wild-type IL-13 conjugated thereto, the liposome encapsulating an anti-cancer drug.

33. The liposome of claim 32, wherein the liposome comprises:
distearophosphoethanolamine polyethyleneglycol 2000 (DSPE-PEG), dipalmitoylphosphatidylcholine (DPPC), cholesterol (CHOL), and stearylamine (SA).

34. The liposome of claim 32, wherein the liposome further comprises MCC (1,2-Dipalmitoyl-sn-glycero-3-phosphoethanolamine-N-[4-(p-maleimidomethyl)cyclohexane-carboxamide]) and/or DSPE-PEG-Maleimide (1,2-Distearoyl-sn-Glycero-3-Phosphoethanolamine-N-[Maleimide(Polyethylene Glycol)2000 (Ammonium Salt)).

35. A method of treating and/or diagnosing an actual or suspected CNS disorder in an individual, comprising:
administering a therapeutically effective amount of a pharmaceutical composition comprising a plurality of particulate delivery vehicles, each associated with a cargo moiety which is a therapeutic and/or diagnostic agent, wherein the association of the therapeutic and/or diagnostic agent with the plurality of particulate delivery vehicles facilitates passage of the therapeutic and/or diagnostic agent through the blood brain barrier into the CNS such that the actual or suspected CNS disorder is treated and/or diagnosed.

36. The method of claim 35, wherein the CNS disorder is cancer.

37. The method of claim 35, wherein the particulate delivery vehicle comprises:
distearophosphoethanolamine polyethyleneglycol 2000 (DSPE-PEG), dipalmitoylphosphatidylcholine (DPPC), cholesterol (CHOL), and stearylamine (SA).

38. The method of claim 36, wherein the particulate delivery vehicle further comprises MCC (1,2-Dipalmitoyl-sn-glycero-3-phosphoethanolamine-N-[4-(p-maleimidomethyl)cyclohexane-carboxamide]) and/or DSPE-PEG-Maleimide (1,2-Distearoyl-sn-Glycero-3-Phosphoethanolamine-N-[Maleimide(Polyethylene Glycol)2000 (Ammonium Salt)).

39. The method of claim 35, wherein the particulate delivery vehicles further comprise a targeting moiety.

40. The method of claim 39, wherein the targeting moiety comprises IL-13 and/or a mutant thereof.

41. The method of claim 39, wherein the targeting moiety comprises an IL-13.K105R mutant of human IL-13, and/or an IL-13.E13K mutant of human IL-13.

42. The method of claim 35, wherein the cargo moiety comprises an anti-transferrin siRNA.

43. The method of claim 35, wherein the cargo moiety comprises iron.

44. The method of claim 35, wherein the cargo moiety comprises an anti-cancer compound.

45. A pharmaceutical composition comprising:
a particulate delivery vehicle having a wall, the wall defining an external surface and an internal volume; and,
a cargo moiety associated with the delivery vehicle.

46. The pharmaceutical composition of claim 45, wherein the delivery vehicle is a liposome.

47. The pharmaceutical composition of claim 46, wherein the liposome comprises:
distearophosphoethanolamine polyethyleneglycol 2000 (DSPE-PEG), dipalmitoylphosphatidylcholine (DPPC), cholesterol (CHOL), and stearylamine (SA).

48. The pharmaceutical composition of claim 47, wherein the particulate delivery vehicle further comprises MCC (1,2-Dipalmitoyl-sn-glycero-3-phosphoethanolamine-N-[4-(p-maleimidomethyl)cyclohexane-carboxamide]) and/or DSPE-PEG-Maleimide (1,2-Distearoyl-sn-Glycero-3-Phosphoethanolamine-N-[Maleimide(Polyethylene Glycol)2000 (Ammonium Salt)).

49. The pharmaceutical composition of claim 45, wherein the cargo moiety is at least partially localized in the internal volume of the delivery vehicle.

50. The pharmaceutical composition of claim 45, further comprising a targeting moiety conjugated to the external surface of the wall of the delivery vehicle.

51. The pharmaceutical composition of claim 45, wherein the targeting moiety is a ligand of a receptor present on a target cell.

52. The pharmaceutical composition of claim 45, wherein the receptor is a human IL-13Rα2 receptor.

53. The pharmaceutical composition of claim 45, wherein the targeting moiety is a mutant of IL-13 which binds a human IL-13α2 receptor.

54. A pharmaceutical composition comprising:
a liposome;
targeting moiety; and,
a chemotherapeutic agent.

55. The pharmaceutical composition of claim 54, wherein the liposome comprising DPPC:CHOL:DSPE-PEG:PDP-SA in a ratio of 10:5:1.5:1.5.

56. The pharmaceutical composition of claim 54, wherein the liposome is about 20 to 220 nm in size.

57. The pharmaceutical composition of claim 54, wherein the targeting moiety comprises a thiolated group.

58. The pharmaceutical composition of claim 54, wherein the targeting moiety comprises pyridyl disulphide groups.

59. The pharmaceutical composition of claim 54, wherein the chemotherapeutic agent is cytotoxic for abnormal cells.

60. A method of treating a cancer patient comprising:
administering to the patient a composition comprising pharmaceutical composition comprising:
a liposome;
targeting moiety; and,
a chemotherapeutic agent; and,
treating a cancer patient.

61. The method of claim 60, wherein the liposome comprises distearophosphoethanolamine polyethyleneglycol 2000 (DSPE-PEG), dipalmitoylphosphatidylcholine (DPPC), cholesterol (CHOL), and stearylamine (SA).

62. The method of claim 60, wherein the liposome further comprises MCC (1,2-Dipalmitoyl-sn-glycero-3-phosphoethanolamine-N-[4-(p-maleimidomethyl)cyclohexane-carboxamide]) and/or DSPE-PEG-Maleimide (1,2-Distearoyl-sn-Glycero-3-Phosphoethanolamine-N-[Maleimide(Polyethylene Glycol)2000 (Ammonium Salt)).

63. The method of claim 60, wherein the targeting moiety targets abnormal cells.

64. The method of claim 60, wherein the chemotherapeutic agent is cytotoxic for abnormal cells.

65. The method of claim 60, wherein the pharmaceutical composition can be administered in conjunction with chemotherapy and radio therapy.
